SEQUENCE LISTING (14 sequences)

SEQ ID NO: 1 - DNA, 21 bp, Artificial Sequence
  Description of Artificial Sequence Synthetic primer
ctgggaccaccttatattccc

SEQ ID NO: 2 - DNA, 24 bp, Artificial Sequence
  Description of Artificial Sequence Synthetic primer
tagacccaatatcaggagactagg

SEQ ID NO: 3 - DNA, 27 bp, Artificial Sequence
  Description of Artificial Sequence Synthetic probe
ctagggacaggattggtgacagaaaag

SEQ ID NO: 4 - DNA, 23 bp, Artificial Sequence
  Description of Artificial Sequence Synthetic probe
ttaatgtggctctggttctgggt

SEQ ID NO: 5 - DNA, 21 bp, Artificial Sequence
  Description of Artificial Sequence Synthetic probe
ccactagggacagaaaagccc

SEQ ID NO: 6 - DNA, 23 bp, Artificial Sequence
  Description of Artificial Sequence Synthetic probe
tgtcaccaatctgtccctagtgg

SEQ ID NO: 7 - DNA, 25 bp, Artificial Sequence
  Description of Artificial Sequence Synthetic probe
tctgtcattaatcctgtccctaggg

SEQ ID NO: 8 - DNA, 20 bp, Artificial Sequence
  Description of Artificial Sequence Synthetic primer
caaggactcaaacccagaag

SEQ ID NO: 9 - DNA, 16 bp, Artificial Sequence
  Description of Artificial Sequence Synthetic primer
tggccgcctctactcc

SEQ ID NO: 10 - DNA, 25 bp, Artificial Sequence
  Description of Artificial Sequence Synthetic probe
agatagcactggggactctttaagg

SEQ ID NO: 11 - DNA, 23 bp, Artificial Sequence
  Description of Artificial Sequence Synthetic probe
gataaggaatctgcctaacagga

SEQ ID NO: 12 - DNA, 17 bp, Artificial Sequence
  Description of Artificial Sequence Synthetic probe
cccatccttaggcctcc

SEQ ID NO: 13 - DNA, 29 bp, Artificial Sequence
  Description of Artificial Sequence Synthetic probe
tcctagtctcctgatattgggtctaaccc

SEQ ID NO: 14 - DNA, 20 bp, Artificial Sequence
  Description of Artificial Sequence Synthetic oligonucleotide
ggggccactagggacaggat

(19) United States
(12) Patent Application Publication
(10) Pub. No.: US 2021/0246505 A1
(43) Pub. Date: Aug. 12, 2021

(54) COMPOSITIONS & METHODS FOR MONITORING DNA REPAIR

(71) Applicant: MEMORIAL SLOAN KETTERING CANCER CENTER, New York, NY (US)

(72) Inventors: Daniel Smith Higginson, New York, NY (US); Suleman Salim Hussain, New York, NY (US)

(21) Appl. No.: 17/254,526

(22) PCT Filed: Jun. 21, 2019
(86) PCT No.: PCT/US19/38527

§ 371 Date: Dec. 21, 2020

Related U.S. Application Data
(60) Provisional application No. 62/688,350, filed on Jun. 21, 2018.
(60) Provisional application No. 62/688,864, filed on Jun. 22, 2018.
(60) Provisional application No. 62/800,554, filed on Feb. 3, 2019.

(51) Int. Cl. C12Q 1/6876; C12Q 1/44

(52) U.S. Cl. CPC C12Q 1/6876; C12Q 1/6869; C12Q 1/44

(57) ABSTRACT

The present invention relates to DNA signatures that are characteristic of DNA that has been repaired in a cell by double strand break repair (DSBR) and that furthermore are characteristic of the specific cellular mechanism used to repair the DNA—such as homologous recombination (“HR”), non-homologous end joining (“NHEJ”), or microhomology-mediated end-joining (“MMEJ”). The present invention provides methods for identifying such DNA signatures, and also provides certain identified DNA signatures that are characteristic of DNA repair by HR, NHEJ, or MMEJ. The present invention also provides various methods and compositions that can be used to determine the presence of such DNA signatures in the genomes of living cells.

### CROSS-REFERENCE TO RELATED APPLICATIONS

This application claims the benefit of priority of U.S. Provisional Patent Application No. 62/688,350 filed on Jun. 21, 2018, U.S. Provisional Patent Application No. 62/688,864 filed on Jun. 22, 2018, and U.S. Provisional Patent Application No. 62/800,554 filed on Feb. 3, 2019, the content of each of which is hereby incorporated by reference in its entirety.

### SEQUENCE LISTING

The instant application contains a Sequence Listing which has been submitted electronically in ASCII format and is hereby incorporated by reference in its entirety. Said ASCII copy, created on Jun. 20, 2019, is named MSKCC_035_WO1_SL.txt and is 3,507 bytes in size.

### INCORPORATION BY REFERENCE

For countries that permit incorporation by reference, all of the references cited in this disclosure are hereby incorporated by reference in their entireties. In addition, any manufacturers' instructions or catalogues for any products cited or mentioned herein are incorporated by reference. Documents incorporated by reference into this text, or any teachings therein, can be used in the practice of the present invention.

### BACKGROUND TO THE INVENTION

DNA damaging agents are a cornerstone of cancer therapy. DNA damaging agents include those whose mechanisms of action involve direct generation of double strand breaks (“DSBs”) in DNA (e.g. ionizing radiation), indirect induction of DSBs through formation of DNA-protein adducts (e.g. chemotherapeutic agents such as etoposide and poly ADP ribose polymerase or “PARP” inhibitors), and/or DNA-DNA inter-strand crosslinks (such as platinum salts, mitomycin-C, and nitrogen mustards). DNA damage, such as that caused by DNA damaging agents, can be repaired by innate cellular DNA repair mechanisms. For example, double strand break repair (“DSBR”) occurs through three dominant pathways: homologous recombination (“HR”), non-homologous end-joining (“NHEJ”), and microhomology-mediated end-joining (“MMEJ”). The degree to which such DSBR pathways are active in cancer cells affects the sensitivity of the cells to treatment with DNA damaging agents. For example, increased DSBR activity in cancer cells can lead to resistance to treatment with DNA damaging agents. Thus, the study of the basic mechanisms and biology of DSBR, and the mechanisms of response and resistance to DNA damaging agents, has been and remains a major area of investigation in cancer research. And in recent years an emerging category of small molecule drugs has been developed specifically to target DSBR factors—in order to overcome resistance to DNA damaging agents. These small molecules include several inhibitors of ataxia-telangiectasia mutated (“ATM”), ATM- and Rad3-related (“ATR”) and DNA-dependent protein kinase catalytic subunit (“DNA-PKcs”)—which are now in clinical development. Tools for assessing DSBR capacity (e.g. in cancer cells) are therefore very useful in the development and testing of DNA damaging agents and agents that modulate DNA repair. Similarly, tools for assessing the DSBR capacity of cancer cells can be useful in determining how best to treat a given cancer, for example by determining which classes of DNA damaging agents or DNA repair agents might be most effective for a given situation.

While there are currently several methods for monitoring DSBR, the existing methods have significant limitations. For example, such methods generally require the creation of specialized cell lines with integrated plasmids and reporter cassettes, and thus are not suitable for use with clinical tumor specimens. And these cell-line-based systems assess repair of an exogenous plasmid—which is not necessarily representative of repair of a DSB in the genome of a cell. In addition, the existing methods can only assess repair by one DSBR mechanism at a time. Also, current methods are not able to distinguish between DSBR resulting from non-homologous end-joining (NHEJ) and that resulting from microhomology-mediated end-joining (MMEJ). Furthermore, current methods for assessing DSBR by homologous recombination (such as LST Score, Myriad Genetics Test, HRDetect) cannot detect homologous recombination deficiencies arising from epigenetic silencing or reversion of homologous recombination due to PARP inhibitor resistance. The present invention overcomes these deficiencies in prior methods for assessing double stranded break repair.

### SUMMARY OF THE INVENTION

Some of the main aspects of the present invention are summarized below. Additional aspects are described in the Detailed Description of the Invention, Examples, Drawings, and Claims sections of this disclosure. The description in each section of this patent disclosure, regardless of any heading or sub-heading titles, is intended to be read in conjunction with all other sections. Furthermore, the various embodiments described in each section of this disclosure can be combined in various different ways, and all such combinations are intended to fall within the scope of the present invention.

The present invention provides new methods and compositions for the assessment of double strand break repair (DSBR). These new methods and compositions are summarized below and are also described in more detail in the “Detailed Description” and “Examples” sections of this patent specification.

The new compositions and methods provided herein provide several important advantages over prior methods of assessing DSBR, including that they: (a) can be used to profile DSBR pathway usage and capacity in any live cell, (b) can detect, and distinguish between, DSBR resulting from each of the three major DSBR pathways (i.e. by HR, NHEJ and MIVIEJ) simultaneously at a single genomic DSB site, (c) enable detection of HR-defective cancers (which may be helpful in guiding treatment decisions, as HR-defective cancers are more sensitive to treatment with crosslinking agents and PARP inhibitors), (d) enable detection of NHEJ and MMEJ deficient cancers (which may be also helpful in guiding treatment decisions, as HEJ and MMEJ deficient cancers are more sensitive to treatment with radiation and RAD51, PARP and DNA-PKcs inhibitors), (e) enable verification of target engagement of DNA repair inhibitors in living cells (e.g. in drug screening settings and in preclinical and clinical testing), and (f) facilitate basic mechanistic research and discovery of DNA repair pathway usage and deficiencies, for example in cancer.

The new methods described herein involve inducing a double strand break (DSB) at a specific chosen site in a cell's genomic DNA using an endonuclease (such as, for example, the Cas9 endonuclease), and subsequently analyzing the repaired DNA (i.e. following repair of the double strand break) to detect “genetic scars” in the repaired DNA that serve as DSBR signatures and that are characteristic of, and specific to, DSBR resulting from either NHEJ, NHEJ or HR. In some embodiments the methods described herein enable one to identify novel DSBR signatures. In other embodiments the present invention provides DSBR signatures that have been identified using such methods and that are common to DNA that has been repaired by DSBR (regardless of the precise gene/location of the original DSB) but that are specific to the particular pathway by which the DSB was repaired—i.e. a signature that is specific to repair by NHEJ, a signature that is specific to repair by NHEJ and a signature that is specific to repair by HR. And in yet other embodiments the present invention provides specific DSBR signatures (and primers and probes useful for the detection of these specific DSBR signatures) that are specific to repair of a specific double strand break (DSB) at a specific location within the “safe-harbor” site on chromosome 19 (locus PPP1R12C) of the human genome called AAVS1, and that are also specific to the particular pathway by which that DSB in the AAVS1 safe harbor locus is repaired (i.e. NHEJ, NHEJ or FIR). The non-gene-specific methods described herein (i.e. those that are not specific to the AAVS1 locus) can be used to study DSBR at any desired location in the genome. The gene-specific methods described herein are designed for studying DSBR at one specific location in the genome—i.e. the AAVS1 locus. Both the non-gene-specific and gene-specific methods have a wide variety of applications. For example, the methods can be used to study the biology of the DSBR process, to assess the DSBR process in any desired cell type (e.g. in any desired cancer cell or clinical cancer specimen), to screen for agents that affect the DSBR process (e.g. DSBR inhibitors), or to assess the effect of any desired agent on the DSBR process (whether in vitro, in ex vivo cell samples, in preclinical studies, or in clinical studies, etc.). In particular, it should be noted that, while the AAVS1-specific methods described herein assess DSBR at one specific locus only, these methods are nonetheless widely applicable to studying DSBR in a wide variety of situations—just as for non-gene-specific methods.

Accordingly, in one embodiment the present invention provides a method of identifying double strand break repair (DSBR) sequence signatures in DNA that has undergone repair of a double strand break (DSB), wherein the DSBR sequence signatures are characteristic of, and specific to, the cellular pathway by which the DSB was repaired, the method comprising: a) contacting the genome of cells of interest and the genome of control cells with an endonuclease to create blunt-ended DSBs at a predetermined location in the genomes of the cells of interest and the control cells, wherein the control cells are deficient in (i) the homologous recombination (HR) DSBR pathway (HR control cells), (ii) the nonhomologous end-joining (NHEJ) DSBR pathway (NHEJ control cells), or (iii) the microhomology-mediated end-joining (MMEJ) repair pathway (MMEJ control cells), b) determining the DNA sequences of the genomic DNA from the cells of interest and from the control cells in the region spanning the location of the DSBs after the cells have been maintained in culture for sufficient time to allow repair of the DSBs by the cells' innate DNA repair machinery, and c) comparing the DNA sequences identified in step (b) from the cells of interest to those of the HR control cells, the NHEJ control cells and/or the MMEJ control cells, wherein: (i) sequence signatures that differ between the cells of interest and the HR control cells are HR DSBR signature sequences, (ii) sequence signatures that differ between the cells of interest and the NHEJ control cells are NHEJ DSBR signature sequences, and (iii) sequence signatures that differ between the cells of interest and the MMEJ control cells are MMEJ DSBR signature sequences. In some variations of this embodiment controls cells need not be used, for example if the sequences of the genomic DNA in control cells have previously been determined, in which case the DNA sequences identified in step c) can be compared to such control sequences.

In another embodiment the present invention provides a method of assessing the activity and/or usage of homologous recombination (HR), nonhomologous end-joining (NHEJ) and/or microhomology-mediated end-joining (MMEJ) DSBR repair pathways in cells of interest, the method comprising: a) contacting the genome of cells of interest with an endonuclease to create blunt-ended double strand breaks (DSBs) at a predetermined location in the genome of the cells of interest, and b) after the cells have been maintained in culture for sufficient time to allow repair of the DSBs by the cells' innate DNA repair machinery, determining whether the repaired DNA comprises an HR-specific DSBR signature sequence, an NHEJ-specific DSBR signature sequence, or an MMEJ DSBR signature sequence, in the region of the cells' genome spanning the DSB location, wherein, if the repaired DNA comprises an HR-specific DSBR signature sequence then the HR DSBR pathway is active in the cells of interest, and if the repaired DNA comprises a NHEJ-specific DSBR signature sequence then the NHEJ DSBR pathway is active in the cells of interest, and if the repaired DNA comprises an MMEJ-specific DSBR signature sequence then the MMEJ DSBR pathway is active in the cells of interest.

In another embodiment the present invention provides a method of assessing the activity and/or usage of homologous recombination (HR), nonhomologous end-joining (NHEJ) and/or microhomology-mediated end-joining (MMEJ) DSBR repair pathways in cells of interest by sequencing, the method comprising: (a) determining the DNA sequence of a genomic region spanning a repaired double strand break in a cell of interest (test DNA sequence), and (b) comparing the test DNA sequence to a control DNA sequence, wherein the control DNA sequence comprises a wild-type version of the same genomic region that has not been subjected to a double strand break or to DSB repair, wherein: (i) if the test DNA sequence comprises a 1 bp deletion relative to the control DNA sequence it has been repaired by nonhomologous end-joining (NHEJ), and (ii) if the test DNA sequence comprises a 12 bp deletion with 5 bp of microhomology (MH) relative to the control DNA sequence it has been repaired by MMEJ.

In another embodiment the present invention provides a method of assessing the activity and/or usage of homologous recombination (HR), nonhomologous end-joining (NHEJ) and/or microhomology-mediated end-joining (MMEJ) DSBR repair pathways in cells of interest by sequencing, the method comprising: (a) determining the DNA sequence of a genomic region spanning a repaired double strand break (DSB) in a cell of interest (test DNA sequence), wherein the DSB was between nucleotides 55115755 and 55115754 of human chromosome 19, GRCh38.p12 within the AAVS1 safe-harbor site, and (b) comparing the test DNA sequence to a control DNA sequence, wherein the control DNA sequence comprises a wild-type version of the same genomic region that has not been subjected to a double strand break or to DSB repair, wherein: (i) if the test DNA sequence comprises a 1 bp deletion relative to the control DNA sequence it has been repaired by nonhomologous end-joining (NHEJ), and (ii) if the test DNA sequence comprises a 12 bp deletion with 5 bp of microhomology (MH) relative to the control DNA sequence it has been repaired by MMEJ.

In another embodiment the present invention provides a method of assessing the activity and/or usage of homologous recombination (HR), nonhomologous end-joining (NHEJ) and/or microhomology-mediated end-joining (MMEJ) DSBR repair pathways in cells of interest by PCR, the method comprising: In another embodiment the present invention provides (a) amplifying a genomic region spanning a repaired double strand break (DSB) between nucleotides 55115755 and 55115754 of human chromosome 19, GRCh38.p12 (within the AAVS1 safe-harbor site) from a test cell of interest by PCR to generate test PCR products, and (b) optionally amplifying the same genomic region that has not been subjected to a double strand break or to DSB repair from a control cell to generate control PCR products, and (c) contacting the test PCR products and optionally the control PCR products with (i) a MMEJ-specific probe comprising SEQ ID NO. 5 and/or (ii) a NHEJ-specific probe comprising SEQ ID NO. 6, and/or (iii) a HR-specific probe comprising SEQ ID NO. 7.wherein, if the MMEJ-specific probe binds to the test product PCR product then the MMEJ DSBR pathway is active in the cells of interest, and/or if the MMEJ-specific probe binds to the test product PCR product then the MMEJ DSBR pathway is active in the cells of interest, and/or if the HR-specific probe binds to the test product PCR product then the HR DSBR pathway is active in the cells of interest.

In other embodiments the present invention provides various kits and compositions that may be useful in carrying out the various methods described herein. For example, in some such embodiments the present invention provides a kit or a composition for assessing the activity and/or usage of homologous recombination (HR), nonhomologous end-joining (NHEJ) and/or microhomology-mediated end-joining (MMEJ) DSBR repair pathways for repair of a double strand break (DSB) between nucleotides 55115755 and 55115754 of human chromosome 19, GRCh38.p12 (within the AAVS1 safe-harbor site), wherein the kit or composition comprises:(a) a forward PCR primer comprising SEQ ID NO. 1, (b) a reverse PCR primer comprising SEQ ID NO. 2, (c) a MMEJ-specific probe comprising SEQ ID NO. 5, a NHEJ-specific probe comprising SEQ ID NO. 6, and, optionally a HR-specific probe comprising SEQ ID NO. 7. In some embodiments the kit or composition comprises one of the following components: (a) a MMEJ-specific probe comprising SEQ ID NO. 5, (b) a NHEJ-specific probe comprising SEQ ID NO. 6, and (c) a HR-specific probe comprising SEQ ID NO. 7. In some embodiments two of such components are included in the kit or composition. In some embodiments all three of such components are included in the kit or composition.

In those embodiments described above or elsewhere herein that involve identifying DSBR signature sequences in cells, or assessing the activity and/or usage of different DSBR repair pathways in cells, the cells can be any desired cell type. In some embodiments the cells are cancer cells. In some embodiments the cells are peripheral blood mononuclear cells (PBMCs). In some embodiments the cells are obtained from a biopsy sample. In some embodiments the cells are obtained from a patient-derived xenograft (PDX). In some embodiments the cells are human cells.

In those embodiments described above or elsewhere herein that involve an endonuclease, in some embodiments the endonuclease is any endonuclease capable of making blunt ended double strand breaks in DNA. In some embodiments the endonuclease is a Cas9 endonuclease. In some embodiments the cells are transfected with a sequence that encodes the endonuclease, such as Cas9. In some embodiments the cells are transfected with a vector that encodes Cas9 and that also includes a guide RNA (gRNA) cassette, wherein the gRNA is specific for the predetermined location in the genome into which a double strand break (DSB) is to be introduced. In some embodiments the cells are transfected with the pX330 vector.

In those embodiments described above or elsewhere herein that involve creating a DSB at a predetermined location in the genome, in some embodiments any desired location in the genome can be used. In some embodiments the predetermined location is within the AAVS1 safe-harbor site on human chromosome 19 (locus PPP1R12C). In some embodiments the predetermined location is is between nucleotides 55115755 and 55115754 of human chromosome 19, GRCh38.p12.

In those embodiments described above or elsewhere herein that involve identifying a homologous recombination (HR) specific sequence signature or assessing the activity and/or usage of HR DSBR repair pathways in cells, typically such embodiments involve transfecting the cells with a donor template for homologous recombination, wherein the donor template comprises one or more mutations that are not present at the predetermined location in the genome of the cells into which a DSB is to be introduced. The donor template can be transfected alone as an oligonucleotide or within vector. In some embodiments the HR donor template is in the same vector as the endonuclease (e.g. Cas9) and the guide RNA. DNA that comprises these mutations can be detected (e.g. by sequencing or by hybridization with a probe)—thereby demonstrating that the repair occurred by homologous recombination. In such embodiments, any suitable mutations that can be used to distinguish the HR-repaired DNA from other DNA (e.g. the corresponding uncut DNA) can be used.

Some of the embodiments described above or elsewhere herein involve using control cells in which a key component of one or more DSBR pathways has been inactivated or inhibited—either genetically or pharmacologically. In some of such embodiments a key component of the HR DSBR pathway has been knocked out genetically and/or inhibited pharmacologically. In some of such embodiments a key component of the NHEJ DSBR pathway has been knocked out genetically and/or inhibited pharmacologically. In some of such embodiments a key component of the MMEJ DSBR pathway has been knocked out genetically and/or inhibited pharmacologically. In each case the result is that that specific DSBR pathway is not functional in the cell, or not functional to a detectable level, or inhibited to a degree that is only active at about 10% or less of its normal level (i.e. its level in the absence of the knockout or pharmacological inhibition). The Examples section of this patent application describes several key components of each DSBR pathway that can be knocked out genetically or pharmacologically inhibited. The Examples section of this patent application also describes several cell lines in which such components are genetically knocked out, and several agents that can pharmacologically inhibit such components.

In those embodiments described above or elsewhere herein that involve determining a DNA sequence, any suitable methods for determining the sequence of DNA can be used. In some embodiments next generation sequencing (NGS) methods are used. In some such embodiments NGS sequencing methods are used to determine DNA sequences resulting from more than 1,000 different repair events, or more than 10,000 different repair events, or more than 100,000 different repair events, or more than 200,000 different repair events in a given sample or in a given experiment.

In those embodiments of the present invention described above or elsewhere herein that involve creating a DSB in DNA and then assessing repair of that DSB, repair must be assessed a sufficient amount of time after creation of the DSB such that repair of the DSB (or a significant amount of repair of the DSB) has occurred. In some embodiments repair is assessed about 2 days, or about 3 days, or about 4 days, or about 5 days, or more after creation of the DSB. Typically, the cells will be maintained in culture during that period of time, i.e. during the repair period.

In those embodiments of the present invention described above or elsewhere herein that involve PCR, any suitable PCR system can be used. In some embodiments the PCR is quantitative PCR. In some embodiments the PCR is digital droplet PCR. In some embodiments a PCR reaction is performed to generate PCR products that span the location of the DSB that has been created and/or repaired. In some embodiments such PCR products are approximately 500 bp long. In some embodiments such PCR products are approximately 400 bp long. In some embodiments such PCR products are approximately 300 bp long. In some embodiments such PCR products are approximately 200 bp long. In some embodiments such PCR products are approximately 100 bp long.

In those embodiments described above or elsewhere herein that involve identifying DSBR signature sequences in cells, or assessing the activity and/or usage of different DSBR repair pathways in cells, in some embodiments such methods can be performed in a quantitative manner to allow quantification of the DSBR signature sequences and/or quantification of the number of repair events or sequence reads comprising such DSBR signature sequences (e.g. HR DSBR signature sequences, NHEJ DSBR signature sequences, and MMEJ DSBR signature sequences.) Each of the methods described herein can easily be performed to allow such quantitative assessment—for example by using high throughput and/or NGS sequencing methods, quantitative PCR, digital droplet PCR, etc. Numerous examples of performing the methods of the invention in a quantitative manner are described in the Examples section of this patent application. Such methods allow quantitative assessment of DSBR pathway usage in different situations (e.g. in different cell types, in the presence or absence of inhibitors or candidate inhibitors of DSBR, etc.)

In those embodiments described above or elsewhere herein that involve primers and/or probes, in some embodiments the primers and/or probes comprise one or more detectable moieties, such as fluorescent labels, fluorophores having a fluorescence property that changes upon hybridization, or fluorophores and quenchers. Suitable primers and probes can be readily designed based on the intended application. Exemplary primers and probes that can be used in the PCR amplification and/or detection of DSBR products in the AAVS locus are provided in Table 1 in Example 1 and include SEQ ID NO.s 1-13.

While some of the main embodiments of the present invention are summarized above, additional embodiments and additional details are provided and described in the Brief Description of the Figures, Detailed Description of the Invention, Examples, Claims, and Figures sections of this patent application. Furthermore, it should be understood that variations and combinations of each of the embodiments described above and elsewhere herein are contemplated and are intended to fall within the scope of the present invention.

### BRIEF DESCRIPTION OF THE FIGURES

FIG. 1A-I. Validation of DSBR-seq. FIG. 1A-D: Histograms demonstrating the frequency of deletion per position surrounding a DSB. FIG. 1A-B: DNApkcs inhibition, LIG4 k.o. increases the size of deletions. FIG. 1C-D: POLQ k.o. and palbociclib treatment decreases the size of deletion. FIG. 1E-F: The top 20 most common specific reads are ranked by size and heat map represents the % of mutant reads. The deletion size and length of microhomology(MH) are shown. FIG. 1E: DNA-pk inhibition or LIG4 k.o. decreases a 1 bp deletion read and increases a 12 bp deletion read with 5 bp of MH. FIG. 1F: POLQ k.o. and palbociclib suppress the 12 bp deletion and increase the 1 bp read. FIG. 1G: POLQ loss decreases MMEJ and increases HR. FIG. 1H: LIG4 loss decreases NHEJ and increases MMEJ and HR. FIG. 1I: DNApkcs inhibitor suppresses NHEJ and increases MMEJ and HR.

FIG. 2. Principal component analysis (PCA1 vs PCA2) using WT, LIG4 k.o., and POLQ k.o. cell lines in the HAP1 background. Specific reads driving MMEJ & NHEJ are shown.

FIG. 3. The characteristic 1 bp del (NHEJ) and 12 bp del (MMEJ) is observed across several cell types using DSBR-Seq. The names of each cell type/cell line are shown.

FIG. 4. Schematic of DSBR-ddPCR. Ref probe (HEX signal) is the loading control. NHEJ, MMEJ, HR and SSA probes (FAM signal) will bind to each of their respective pathway characteristic reads.

FIG. 5 A-I. DSBR-ddPCR shows similar results to DSBR-Seq. FIG. 5A—WT-drop off to calculate the mutant fraction. Ref and WT probe bind equally in un-transfected (UT) samples while in Cas9 cut samples the WT probe binding is lower than Ref, confirming the cut in the DNA and this drop-off is used to calculate the mutant fraction for each sample. FIG. 5B-C—Optimization of the MMEJ probe using MMEJ ssDNA positive control. The MMEJ probe only binds to the Cas9 cut DNA and not in un-transfected samples. FIG. 5D-E—Validation of MMEJ probes showing decreased MMEJ after palbociclib treatment and in Hap1 PolQ KO cells. FIG. 5F-G—Validation of NHEJ probes using the Hap1 Lig4 KO and in HEK29T cells treated with 1 μM DNAPk inhibitor (NU-7441). FIG. 5H-I—Validation of HR probe in BRCA1 deficient (pcDNA) and BRCA1-complemented (BRCA1) HCC1937 cells and in Hap1 Lig4 KO cells.

FIG. 6. Schematic of technique to identify characteristic DSBR pathway profiles, which can be used as an assay endpoints for repair (DSBR-seq). Highly characteristic reads can also be measured with dedicated ddPCR primer/probes (DSBR-ddPCR).

FIG. 7. Schematic Representation of key features of different DSBR pathways.

FIG. 8. Newly developed algorithm for detecting microhomology at breakpoints.

FIG. 9. Profile of deletions, insertions and substitutions surrounding a DSB at position 120 bp within the sequenced amplicon.

FIG. 10. Deletion profile by size using HAP1 WT and isogenic LIG4, POLQ, and RAD52 k.o. controls. Reciprocal effects of LIG4 and POLQ k.o. are observed whereas a RAD52 knockout HAP1 line finds little change from the WT condition.

FIG. 11A-C. FIG. 11A. PCA analysis (PCA1 vs PCA2) using WT, LIG4 k.o., and POLQ k.o. cell lines in the HAP1 background. Specific reads characteristic of MMEJ and NHEJ are shown. FIG. 11B. Significant PCs represent known and unknown pathways. FIG. 11C. Reciprocity between cNHEJ and MMEJ reads in Lig4 and POLQ knockout cell lines.

FIG. 12A-B. FIG. 12A. DSBR-seq captures the effect of the drug palbociclib on DSBR. The drug results in G1 arrest, suppressing HR and MMEJ. Top panels: Cas9 and the donor sequence used. Bottom panels: only cas9 used. Note: peaks at 20 and 180 bp were a result of the index sequence in this experiment. FIG. 12B. Deletion size profile of asynchronous and G1 enriched Hap1 cells which were treated with palbociclib. MMEJ reads are suppressed.

FIG. 13. Example of how DSBR-seq can be used to discover relationships between cancer specific alterations and DSBR pathway choice. Top panels—HPV16 E7 vs. empty vector transiently expressed in U2OS cell lines with stably integrated reporter cassettes. Bottom panels—DSBR-seq results in the U2OS cell line transfected with HPV16 E7 expression or empty vector.

FIG. 14. Schematic representation of exemplary DSBR-ddPCR method.

FIG. 15. Schematic representation of a DSBR-seq method according to the invention involving steps for transfection of cells with Cas9, guide RNA (gRNA) and donor template (for homologous recombination), generation of double-strand breaks (DSBs), repair of double strand breaks (DSBR), extraction of genomic DNA following DSBR, sequencing of the repaired DNA using next generation sequencing, and bioinformatics analysis to identify and/or detect DSBR signatures specific to different DSBR pathways (i.e. HR, NHEJ, MMEJ/Alt-EJ).

FIG. 16. Schematic representation of a DSBR-PCR (DSBR-ddPCR) method according to the invention. The initial steps of the method are the same as those for DSBR-seq shown in FIG. 15 (i.e. from transfection of cells to extraction of genomic DNA), but instead of characterizing the repaired DNA by sequencing, PCR (e.g. ddPCR) is performed and probes specific for HR, NHEJ and MMEJ/ALT-EJ-generated DSBR products are used to detect and/or quantify each of these pathway-specific DSBR products. PCR products (i.e. amplicons) spanning the break site are generated, and the various different probes illustrated schematically in FIG. 16 are used to detect and/or quantify NHEJ and MMERALT-EJ-generated DSBR products. Wild-type (WT) and reference (Ref) probes may also be used (see FIG. 4).

### DETAILED DESCRIPTION

The sub-headings provided below, and throughout this patent disclosure, are not intended to denote limitations of the various aspects or embodiments of the invention, which are to be understood by reference to the specification as-a-whole. For example, this Detailed Description is intended to read in conjunction with, and to expand upon, the description provided in all other sections of this patent application, including the Summary of the Invention and Examples sections thereof.

Definitions & Abbreviations

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Units, prefixes, and symbols are denoted in their Systeme International de Unites (SI) accepted form. Numeric ranges provided herein are inclusive of the numbers defining the range.

Where a numeric term is preceded by “about” or “approximately” the term includes the stated number and values ±10% of the stated number.

Wherever embodiments are described with the language “comprising,” otherwise analogous embodiments described in terms of “consisting of” and/or “consisting essentially of” are included.

The abbreviation “DSB” refers to double strand breaks.

The abbreviation “DSBR” refers to double strand break repair.

The abbreviation “HR” refers to homologous recombination.

The abbreviation “NHEJ” refers to non-homologous end-joining.

The abbreviation “MMEJ” refers to microhomology-mediated end-joining.

The abbreviation “Alt-EJ” refers to alternative end-joining—which is the same as MMEJ.

The terms Alt-EJ and MMEJ may be used interchangeably herein.

The abbreviation “k.o.” refers to knockout.

The phrases “signature sequence” and “sequence signature” may be used interchangeably herein.

Various other terms and abbreviations are defined elsewhere in this patent disclosure, where used. Furthermore, terms that are not specifically defined herein may be more fully understood in the context in which the terms are used and/or by reference to the specification in its entirety. Where no explicit definition of a term is provided, or is clear from the context in which the term is used, such terms have the meanings commonly understood by those of ordinary skill in the art to which this invention pertains.

II. Cells & Tissue Samples

The methods and compositions described herein can be used to interrogate the DSBR process in any cell of interest. In some embodiments the cell is a human cell. In some embodiments the cell is in vitro. In some embodiments the cell is ex vivo. In some embodiments the cell is in a patient-derived xenograft (PDX). In some embodiments the cell is in a tissue sample (the terms tissue sample, tissue specimen, patient specimen, biopsy sample, clinical sample, and clinical specimen are used interchangeably herein). In some embodiments the cell is in culture—e.g. in a short-term culture obtained by dissociation of a tissue sample. In some embodiments the cell is a cancer cell. In some embodiments the cell is a peripheral blood mononuclear cell (PBMC).

III. Creation of Double Stand Breaks (DSBs), Repair of DSBs, & Characterization of Repaired DSBs by Sequencing (DSBR-seq) and/or PCR (DSBR-PCR)

Several of the embodiments of the present invention involve the creation of double strand breaks (DSBs) at a specific location in the genome of a cell of interest. In some embodiments the break is created using any suitable endonuclease enzyme. In some embodiments the DSB is generated using an endonuclease that generates blunt double strand breaks, such as the Cas9 endonuclease. A particular advantage of using the CRISPR/Cas9 system to make the DSBs is the ease with which the break can be made at a specific predetermined locus in a controllable manner.

The specific location selected for creating a DSB can be any desired location in the genome. In some embodiments the break is made within the “safe-harbor” site on chromosome 19(locus PPP1R12C) of the human genome called AAVS1. In some embodiments the break is made specifically between nucleotides 55115755 and 55115754 of human chromosome 19, GRCh38.p12—i.e. with the AAVS1 safe harbor locus. This specific break in AAVS1 can readily be made by transfecting cells with a commercially available plasmid (Addgene plasmid #72833; http://n2t.net/addgene:72833; RRID:Addgene_72833)—which is referred to herein as the “pX330” plasmid/vector, and which contains expression cassettes for Cas9 and guide RNA (gRNA) for targeting the AAVS1 safe harbor locus in human cells. The gRNA/shRNA sequence in this vector is GGGGCCACTAGGGACAGGAT (SEQ ID NO. 14). See Natsume et al., “Rapid Protein Depletion in Human Cells by Auxin-Inducible Degron Tagging with Short Homology Donors,” Cell Rep. 2016 Apr. 5; 15(1):210-8. (doi: 10.1016/j.celrep.2016.03.001. Epub 2016 Mar. 24) and the description at http://n2t.net/addgene:72833 for further details of this vector (including vector maps), sequences, and protocols for its use, etc.

Repair of the DSBs introduced using the methods of the present invention occurs automatically in the cells of interest—using the cells' innate DNA repair machinery. The repair typically occurs over the course of a few hours to a few days. For example, a suitable level of repair may be achieved about 1 day, or about 2 days, or about 3 days, or about 4 days, or about 5 days after creation of the break, or after transfecting the cells with a vector encoding the break-inducing endonuclease (e.g. Cas9). Typically, in carrying out the methods of the present invention, the cells of interest are left (e.g. maintained in culture) for about 3 days following generation of the DSB (e.g. for about 3 days following transfection with the endonucleases/vectors described above) to allow repair of the DSBs to occur before proceeding with next steps of the methods.

After the DSBs have been repaired to a sufficient degree, typically genomic DNA (gDNA) is extracted from the cells of interest. In some embodiments fragments (typically around 200 bp in length) of the genomic DNA spanning the break site are then amplified by PCR. The repaired gDNA, or amplified PCR products derived therefrom, are then characterized to assess the DSBR process/mechanism This characterization can be carried out in a variety of ways—including, but not limited to, by determining the sequence of the repaired DNA in the vicinity of the DSB, and/or by identifying the presence of HR, NHEJ and/or MMEJ-specific signatures in the repaired DNA either by sequencing, by PCR, or by hybridization of specific probes, or by some combination of such methods. In this way, the usage (or relative usage) of the HR, NHEJ and/or MMEJ DSBR repair pathways in the cells of interest can be assessed.

In some embodiments the repaired DNA is characterized in the vicinity of the break site by sequencing, typically using a high-throughput next generation sequencing (NGS) method to obtain sequence reads of large numbers of different repair events. Bioinformatic analysis can then be used to analyze the sequence reads and identify frequently occurring sequence reads and the characteristics of those reads (e.g. deletions, microhomologies, etc.) that are associated with repair by each DSBR mechanism. New DSBR signature sequences that are characteristic of HR, NHEJ and/or MMEJ-repaired DNA in general can be identified in this way. Similarly, DSBR signature sequences that are characteristic of HR, NHEJ and/or MMEJ-repaired DNA at specific break sites in specific genes can also be identified in this way. A key aspect of these methods that allows the identification of DSBR signature sequences that are characteristic of HR, NHEJ and/or MMEJ-repaired DNA, is the use of appropriate controls in carrying out these methods. For example, and as described at length in the Examples section of this patent application, these methods can be carried out using either or both of two categories of controls: (1) controls in which one or more components of the DSBR machinery for a specific repair pathway have been knocked out from the cells, and/or (2) controls in which one or more components of the DSBR machinery for a specific repair pathway have been inhibited pharmacologically. Another key aspect for the assessment of DSB repair by homologous recombination (which is generally error free) using the methods described herein, is the transfection of the cells with donor template DNA that contains one or more mutations (e.g. substitutions) or other marks or labels that allow one to distinguish DNA that has been repaired by HR from WT uncut DNA.

As described above, and elsewhere herein, the methods described herein enable the identification of DSBR signatures specific for different DSBR pathways—such as HR, NHEJ and MMEJ. Once such signatures have been identified (as they have been here), the need to perform high through put sequencing and/or bioinformatics can be significantly reduced or even eliminated. For example, in some embodiments sequencing can be used to determine if one or more of the pathway-specific DSBR signatures is present—without the need for bio-informatic analysis. And in other embodiments, sequencing need not be performed at all, but, instead, repaired DNA can be interrogated in the vicinity of DSB sites using probes designed to bind specifically to DNA sequences containing the already identified NHEJ, HR or MMEJ-specific DSBR signatures. These probes can be used for detection of these signatures in genomic DNA directly, or, more preferably, in amplified PCR products derived from that genomic DNA. Any suitable PCR amplification system can be used. In some embodiments droplet digital PCR (ddPCR) is used. Such probes can easily be designed based on any DSBR signature sequence that is discovered using the methods of the present invention, or based on any of the DSBR signature sequences specifically described herein. Furthermore, sequences of a series of probes specific for HR, NHEJ and MMEJ-specific repair of a DSB between nucleotides 55115755 and 55115754 of human chromosome 19, GRCh38.p12 i.e. within the AAVS1 locus (as well as various control probes) are specifically provided herein. For example, sequences of such probes, as well as sequences of suitable primers for amplification of a region surrounding the DSB between nucleotides 55115755 and 55115754 of human chromosome 19, GRCh38.p12, are provided in Table 1 in Example 1 and in the Sequence Listing portion of this patent disclosure (see SEQ ID Nos. 1-13).

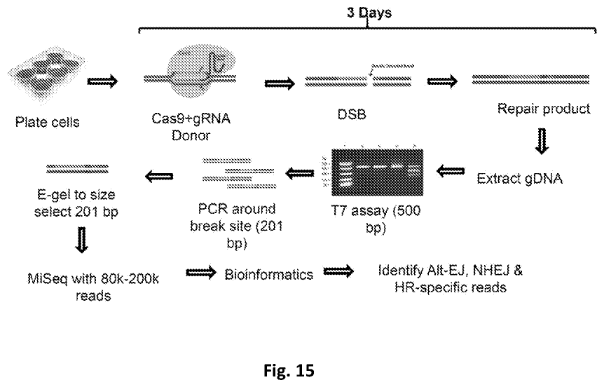

A schematic representation of an exemplary DSBR-seq method according to the present invention is provided in FIG. 15. This exemplary method comprises: (a) transfecting cells of interest with Cas9, guideRNA and donor template (for homolgous recombination)—thereby generating double-strand breaks (DSBs), (b) maintaining the transfected cells in culture for a period of time sufficient to allow double strand break repair to occur (approximately 3 days), (c), extracting genomic DNA from the cells, (d) performing PCR to amplify DNA fragments spanning the break site (here the amplicons are about 20 bp long), (e) performing a next generation sequencing method to obtain a large number of sequence reads for a large number of repair events (in this example approximately 80,000 to 200,000 reads), and (f) performing bioinformatic analysis to identify HR, NHEJ and MMEJ-specific sequence reads/DSBR signatures. This method can be conducted using suitable knock out controls and/or pharmacological controls for each DSBR pathway—as described above and elsewhere herein.

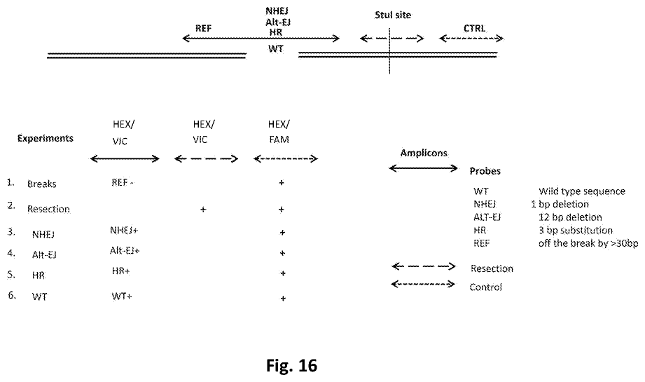

A schematic representation of an exemplary PCR-based method for assessing DSBR pathway usage according to the invention is provided in FIG. 16 The initial steps of the method are largely the same as those for DSBR-seq shown in FIG. 15 (i.e. from transfection of cells to PCR amplification), but then, instead of characterizing the repaired DNA by sequencing, probes specific for HR, NHEJ and MMEJ/ALT-EJ-generated DSBR products are used to detect and/or quantify pathway-specific DSBR products. In some such embodiments any type of PCR amplification can be used. In some embodiments droplet digital PCR (ddPCR) is used. FIG. 16 shows schematically the location to which the various DSBR pathway specific and control probes bind on the amplified DNA, relative to the break site.

An additional schematic representation of an exemplary DSBR-ddPCR assay is shown in FIG.

4. In this example, a Ref probe (HEX signal) is used as a loading control for each droplet containing the amplified PCR product. The WT probe (FAM signal) attaches to uncut WT sequences. In the presence of a mutation introduced by repair of a DSB created by CRISPR-Cas9, the WT probe will not be able to bind but the Ref probe should still bind. Using these probes one can therefore determine/estimate the mutated fraction in each experiment and control for transfection efficiency. The NHEJ, MMEJ and HR-specific probes are used to assay DSBR pathway utilization.

IV. PCR Methods, Primers & Probes

In each of the embodiments summarized above, and described elsewhere herein, that involve a PCR reaction, the PCR method can be any suitable PCR method. In some embodiments the

PCR method is a quantitative PCR method, a real-time quantitative PCR method, a digital PCR method or a droplet digital PCR (ddPCR) method. In preferred embodiments the PCR method is a droplet digital PCR (ddPCR) method.

In some of the methods summarized above, and described elsewhere herein, amplified PCR products are detected with a probe. In some such embodiments the probe comprises a detectable moiety. In some such embodiments the probe comprises a fluorescent label. In some such embodiments the probe comprises a fluorophore having a fluorescence property that changes upon hybridization. In some such embodiments the probe comprises a fluorophore and a quencher.

Oligonucleotide primers for PCR can be designed for amplification of the desired DNA sequences using standard methods that are well known in the art. Similarly, probes capable of binding to (i.e. hybridizing to) specified DNA sequences (such as specified PCR products) can be designed using standard methods well known in the art. Binding of probes to their target sequences by hybridization may be conducted under any suitable conditions that allow specific binding. For example, stringent binding/hybridization conditions may be used to achieve specific binding. By “stringent” is meant conditions under which the probe hybridizes to its specific target sequence to a measurably greater extent than to other unrelated sequences—sufficient to allow specific detection of the target to which the probe is designed to bind.

In some embodiments the present invention provides exemplary primers for PCR amplification (SEQ ID Nos. 1-2) and exemplary probes for detection or DNA, e.g. PCR products (e.g. SEQ ID Nos. 3-13). These primers and probes are described in more detail in the Examples section of this application, in Table 1, and in the Sequence Listing. It should be noted that variations of these precise primer and probe sequences can be used without departing from the spirit of the invention. For example, alternative primers and probes that are longer or shorter than the specified primers or probes can be used. And, in some situations, primer or probes having 1, 2, 3, 4 or 5 substitutions as compare to these primers and probes can be used—as long as the primers or probes retain their stated function (e.g. amplification of the region spanning the specified DSB break point, or binding to the specified HR, NHEJ or MMEH signature sequences.

V. Kits & Compositions

In addition to the various methods described above, and elsewhere herein, in some embodiments the present invention also provides compositions and/or kits comprising any one or more of the primers and probes described herein—i.e. comprising any one or more of SEQ ID NO. 1-13. In certain embodiments, such compositions and/or kits may comprise one or more other components that are useful for and/or compatible with use of such primers and probes, including suitable diluents, buffers, carriers, preservatives, labels, tags, nucleic acid molecules, nucleotides, enzymes (such as polymerase enzymes), and the like. In some embodiments such compositions and/or kits comprise one more positive controls and/or negative controls for use in the methods provided herein.

The invention is further described in the following non-limiting Examples.

### EXAMPLES

### Example 1

### Sequencing-Based and PCR-Based Methods for the Identification of and Analysis of Pathway-Specific DSBR Signatures

Introduction

Homologous recombination (HR) is the most important type of DNA repair during the S and G2 phases of the cell cycle, responding to DSBs created during DNA replication, and thus HR is the most important pathway for repairing damage caused by crosslinking agents (cisplatin, mitomycin C) or agents that cause DNA-protein adducts (etoposide, PARP inhibitors). Cancers with germline or acquired deficiencies in HR exhibit sensitivity to cisplatin and PARP inhibitors, a phenomenon confirmed across multiple malignancies [breast(3, 4), ovarian(5-7), and prostate(8)]. In many tumors, genetic deficiencies in HR genes can be identified, but in other tumors epigenetic silencing of BRCA1 or other unknown mechanisms confer HR deficiency(9). Biomarkers of HR deficiency include biallelic germline mutations in HR genes(9) or measurements of either genomic rearrangements [LST score(10), the Myriad Genetics HRD™ test(11, 12)] or single nucleotide substitution patterns [HRDetect(13)]. However, these genomic biomarkers provide only a static view of a tumor's likely HR capacity, measuring sequence changes developed over many years during carcinogenesis. The mechanisms of resistance to platinum agents and PARP inhibitors can involve genetic reversion of the HR defective allele(14), or loss of 53BP1(15) or other factors that reconstitute HR capacity.(16, 17) There is a clinical need for technologies to assess dynamic HR capacity in real time(2). The methods described herein can be used to assess HR capacity in any cell line or tissue specimen, such as peripheral blood mononucleocytes, patient-derived xenografts, or freshly obtained biopsy samples.

Nonhomologous end-joining (NHEJ) is the most important DNA repair pathway in the response to radiotherapy-induced DNA damage as it is the dominant pathway for most breaks induced by therapeutic radiation. Though genetic defects in NHEJ genes are unusual in cancer, likely owing to their essential nature for cell survival, the wide variance in responsiveness to radiation amongst cancers suggests there exist relative differences in NHEJ capacity. Known examples include a) the effect of androgen receptor signaling in prostate cancer on NHEJ(18), b) 53BP1 haploinsufficiency in gliomas(19), c) suppression of NHEJ usage through downstream gene expression programs activated tyrosine kinases BCR-ABL1 and FLT3/ITD in leukemias(20), and d) observed NHEJ defects in selected bladder(21) and ovarian cancers(22). However, previously NHEJ was difficult to measure directly and thus NHEJ capacity is understudied. The methods described herein provide a transformative cellular analysis tool as they allow for the determination of the usage of NHEJ relative to HR and MMEJ, as well as characterization of the spectrum of deletion events. As an example, we have used the methods described herein to demonstrate that the HPV16 E7 protein suppresses NHEJ and increases MMEJ, providing a mechanism for the known clinical radio-sensitivity of HPV-associated head and neck cancer.

Microhomology-mediated end-joining (MMEJ) is characterized by its use of end-resection and the critical factor polymerase theta (POLQ) and its independence from NHEJ factors. MMEJ mediates resistance to radiotherapy and is an essential pathway in cancers with HR deficiency. Deletions caused by alternative end-joining (Alt-EJ) are larger than those created by NHEJ and frequently involve stretches of microhomology that are involved in an initial annealing step between broken ends. Characteristic deletions with microhomology can be identified in HR deficient cancers(13), likely signifying a key role in carcinogenesis in HR defective backgrounds. In addition, small deletions with microhomology are the defining genomic feature of radiation-induced cancers, indicating the pathway is used extensively in the response to therapeutic radiation(23). However, prior to the present invention it was unclear how to clearly distinguish between NHEJ and MMEJ, as small stretches of microhomology (1-2 bp) can be used by NHEJ and can occur by chance as well. The methods described herein can clearly distinguish between NHEJ and MMEJ and measure their relative usage.

As alluded to above, there are a variety of limitations of prior methods for analyzing DSBR. While there are a number of ways to quantitate the total number of DNA double strand breaks (yH2AX foci, COMET, filter elution, and cytogenetics), it is more difficult to assess the specific DSBR pathway used to repair those breaks. Prior to the present invention DSBR was measured by either visualization of foci for Rad51 (HR) or DNA-PKcs/53BP1 (NHEJ) by immunofluorescence, or by using the plasmid based DR-GFP (HR), EJ2-2 GFP (MMEJ) or EJ5-GFP(NHEJ) reporter cassettes (24-25). However, as foci represent pathway intermediates rather than completed repair, this approach cannot measure pathway defects downstream of the particular protein being visualized. And plasmid-based assays require creation of a specialized cell line with an integrated cassette, and thus cannot be widely used in any cell line or human specimen, limiting applicability. Also, the plasmid-based assays can capture only one pathway each, and lack the ability to study pathway trade-offs.

New Methods for DSBR Assessment & New DSBR Signatures

This Example describes a novel approach to create a single double strand break (DSB) in any transfectable cell, followed by next generation sequencing to evaluate the repair consequences in thousands of reads and identify DSBR signatures (e.g. patterns of deletions and/or insertions) that are characteristic of repair by NHEJ, MMEJ or HR. In this example, a

DSB was created specifically in the AAVS safe harbor locus, and specific DSBR signatures characteristic of DSBR by NHEJ, MMEJ or HR at this locus were identified. However, the same approach can be used to identify DSBR signatures characteristic of repair by NHEJ, MMEJ and/or HR at any other desired location in a genome. Furthermore, certain aspects of the DSBR signatures identified for this specific locus are generalizable to other loci.

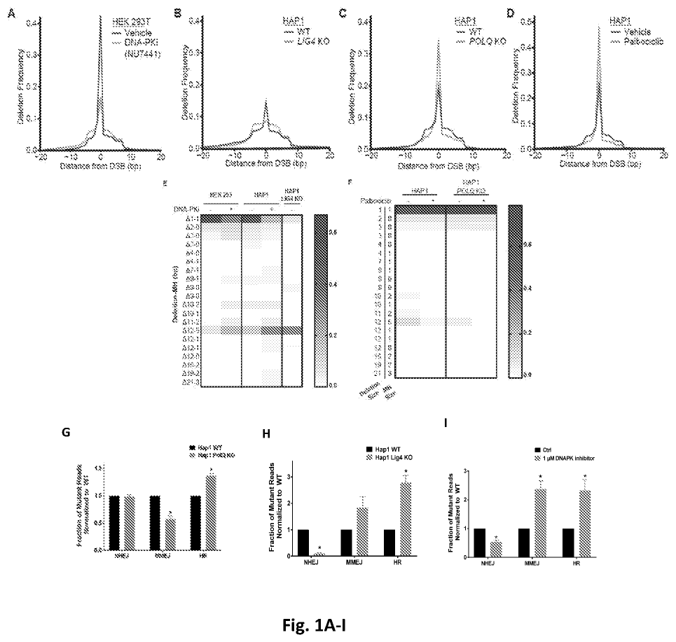

In this Example, transfection of the pX330 plasmid, containing the Cas9 and guide RNA (gRNA) expression cassettes to the AAVS1 safe locus, is used. Genomic DNA (gDNA) is collected 3 days post-transfection. To asses—DNA repair by HR, a donor template containing three substitutions around the protospacer adjacent motif (PAM) which limits cut-recut cycles is transfected also. A 500 bp amplicon surrounding the break is used for rapid measurement of DSBR errors via the widely used T7 endonuclease I assay. A 201 bp amplicon surrounding the cut site, but outside of the HR template is amplified, purified via E-gel electrophoresis system, converted into Illumina sequencing libraries, quantified via qPCR, and then sequenced at 200,000× read depth using paired end 150 bp (PE150) Mi-Seq. Unique, nonsingleton reads (occurring more than once) are aligned to the reference sequence using pairwise alignment (for example using Python). The indels and substitutions are cataloged relative to the break site and microhomology (MH) is determined (for example using an internally developed and validated algorithm, see FIG. 8). The reads are categorized as consequences of NHEJ (≤5 bp deletion) or MMEJ (>5 bp deletion with >2 bp MH). The donor template DNA contains key substitutions—thereby providing a readout of HR. These experiments are also performed in cells that are isogenic knockout controls of key NHEJ, MMEJ and HR components and using pharmacologic controls in which DNA-PKcs, RAD51, and PARP are inhibited physiologically. By comparing the experimental reads against those obtained from the knockout controls and/or pharmacologic controls, the specific reads dependent on each pathway were discovered. (FIG. 1). Thus, these methods involve empirically-determined deletion profiles, derived from known controls deficient in each pathway, as signatures to clearly separate and quantify DSBR events occurring by HR, NHEJ and MMEJ. The single double strand break allows for direct analysis of pathway choice in a native genomic location with a blunt-ended DSB. Because both cell cycle dependent pathways (MMEJ and FIR) are measured in the same experiment, there is an effective built-in control for confounding cell cycle effects on the use of these pathways.

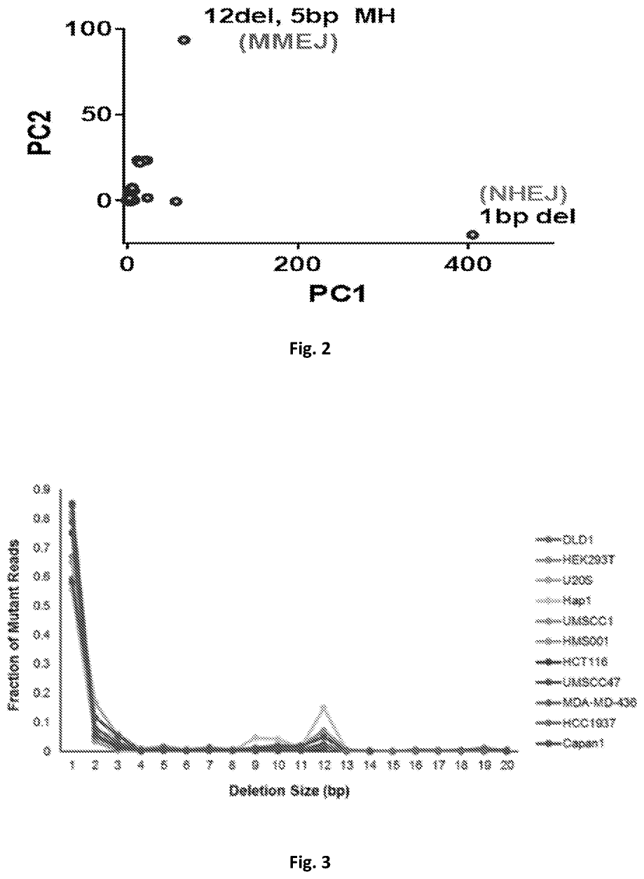

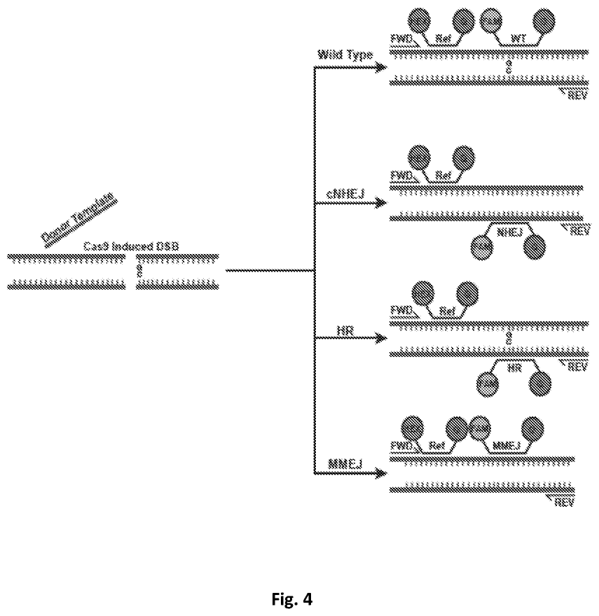

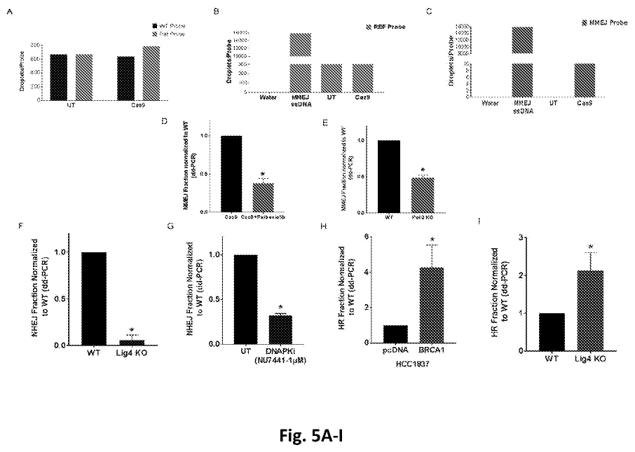

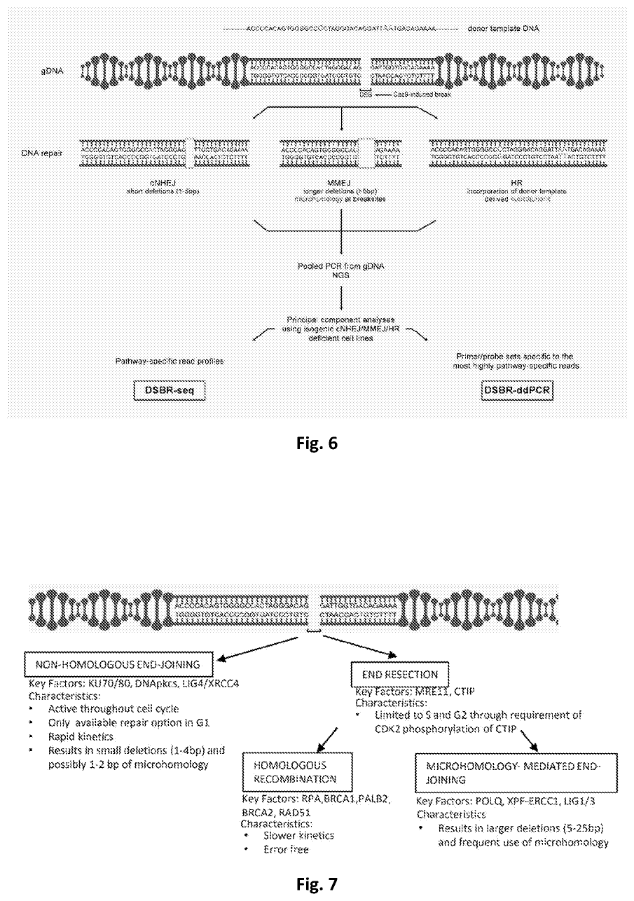

As described above, this method was successfully performed using high throughput next generation sequencing (NGS) (FIG. 1). However, we also wanted to develop a variation of this method that would not require NGS or bioinformatic analysis. Thus, we developed variation on this method in which DSBR signatures specific to each DSBR pathway (i.e. HR, NHEJ and MMEZJ) can be detected using probes that are specific for each DSBR signature. In this example this detection was performed in conjunction with PCR amplification of the repaired genomic regions—in this case using digital droplet PCR (ddPCR). Using principal component analyses, we identified a 1 bp deletion which represents NHEJ and a 12 bp deletion with 5 bp MH which represents MMEJ (FIG. 2). We observed these characteristic 1 bp and 12 bp deletions across multiple cell lines—confirming the wide applicability of these assays (FIG. 3). For optimization of ddPCR probes we used gDNA from the isogenic controls of each pathway from the DSBR-Seq experiments in FIG. 1. We designed a ssDNA positive control for each of the probes, with un-transfected (UT) gDNA to be used as a negative control. The “reference probe” (off the break site) is the loading control for total droplets and the drop-off of “WT probe” (at the break site) is used to estimate the mutant fraction in each experiment and thus control for transfection differences. MMEJ, HR and NHEJ probes may be detected using any suitable system. In this Example they were detected based on a FAM signal (FIG. 4). After optimization with various controls, DSBR-ddPCR for NHEJ, MMEJ and HR mirrored our observations seen with DSBR-Seq. FIG. 5. Thus, DSBR-ddPCR is a rapid technique to assay DSBR. We also designed probes to detect total persistent DSBs and to measure resection. The primers and probe sets we developed and used in these experiments, which are AAVS1-specific—are as follows:

TABLE 1
Sequences of primers and probes for amplification and detection
of human AAVS1-specific sequences
Pathway ddPCR

| Primer Name (SEQ ID NO.) | Sequence |
|---|---|
| Primer.FOR (SEQ ID NO. 1) | CTGGGACCACCTTATATTCCC |
| Primer.REV (SEQ ID NO. 2) | TAGACCCAATATCAGGAGACTAGG |
| Probe Name (SEQ ID NO.) | Sequence |
| WT probe (SEQ ID NO. 3) | CTAGGGACAGGATTGGTGACAGAAAAG |
| Reference Probe (SEQ ID NO. 4) | TTAATGTGGCTCTGGTTCTGGGT |
| MMEJ Probe (SEQ ID NO. 5) | CCACTAGGGACAGAAAAGCCC |
| NHEJ Probe (SEQ ID NO. 6) | TGTCACCAATCTGTCCCTAGTGG |
| HR Probe (SEQ ID NO.7) | TCTGTCAttAATCCTGTCCCTAGgG |

Break analysis ddPCR

| Primer Name (SEQ ID NO.) | Sequence |
|---|---|
| Break Primer 2.FOR (SEQ ID NO. 8) | CAAGGACTCAAACCCAGAAG |
| Break Primer 2.REV (SEQ ID NO. 9) | TGGCCGCCTCTACTCC |
| Probe Name (SEQ ID NO.) | Sequence |
| Break Probe (SEQ ID NO. 10) | AGATAGCACTGGGGACTCTTTAAGG |

Resection ddPCR

| Probe Name (SEQ ID NO.) | Sequence |
|---|---|
| Resection.FOR (SEQ ID NO. 11) | GATAAGGAATCTGCCTAACAGGA |
| Resection.REV (SEQ ID NO. 12) | CCCATCCTTAGGCCTCC |
| Probe Name (SEQ ID NO.) | Sequence |
| Resection Probe (SEQ ID NO. 13) | TCCTAGTCTCCTGATATTGGGTCTAACCC |

### Example 2

Sequencing-based and PCR-based methods for the identification of and analysis of pathway-specific DSBR signatures in human clinical samples

The studies described in Example 1 were performed using human cell lines. However, these methods can also be used in human clinical sample. DSBs can be generated in human clinical samples. For example, Cas9 technology has already been extensively investigated in human tissues allowing ex vivo DNA editing, such as for disorders of the blood like SCID and Fanconi anemia(27-29). Furthermore, introduction of Cas9 into patient-derived xenografts has been achieved with dissociation of tumor cells, transduction of a lentiviral particle, and short-term ex vivo culture(30-31). Thus, the present methods can be used with dissociated biopsy samples.

Tumors defective in HR are more sensitive to DNA damage that requires HR to resolve, such as DNA crosslinks formed by platinum salts or mitomycin and DNA-protein adducts formed by PARP inhibitors or etoposide. Detecting HR deficiency in cancer is the focus of intense investigation and current biomarkers include genomic rearrangement signatures, substitution patterns, or recognized bi-allelic mutations in HR genes. However, resistance to these agents can emerge secondary to genetic reverse events, epigenetic silencing or loss of other genes. Thus, dynamic, functional assays of HR are still needed. The present methods can address these needs. For example, the methods and compositions described herein (e.g. in Example 1) can be used to assess HR and detect HR deficiency tumors—for example using patient-derived xenografts (PDX) or biopsy samples. Similarly, the methods and compositions described herein (e.g. in Example 1) can be used to assess DSBR by NHEJ and MMEJ, and/or deficiencies in these pathways in tumors—for example using patient-derived xenografts (PDX) or biopsy samples.

Another important clinical application of the methods and compositions described herein is in screening for and/or testing potential therapeutic agents such as inhibitors of DSBR pathway components. For example, there is an emerging category of small molecular inhibitors targeting the DNA damage response, but currently there are only limited tools to evaluate target engagement in tumor and normal cells. For instance, M3814, a DNA-PKcs inhibitor, is in phase I trials in combination with ionizing radiation. The compositions and methods described herein for assessment of DSBR by HR, NHEJ and/or MMEJ can be used in clinically-relevant samples or cells (e.g. in PBMCs) to assess target engagement and/or efficacy of such inhibitors.

In proof-of concept studies we have successfully delivered Cas9, gRNA, and donor template DNA to human biopsy samples, achieving a 30% transfection rate with a GFP reporter plasmid using nucleofection (Nucleofector II, AMAX biosystems), and have demonstrated effective genome editing at the AAVS1 locus using the pX330 plasmid and donor template in human-derived, immortalized lymphoblastoid cells (LCLs) (not shown). Thus, the compositions and methods described herein for assessment of DSBR by HR, NHEJ and MMEJ in human clinical biopsy samples—for a variety of different clinical applications.

For example, using this system, the effects of DSBR pathway inhibitors (such as ATM (AZD0156), ATR (AZD6738), DNA-PKcs (M3814), PARP (olaparib), and Rad51(RI-01)) or candidate DSBR pathway inhibitors can be tested in LCLs, PMBCs, or in any other desired cells from any other desired human clinical samples, enabling the effects of these agents on DSBR by HR, NHEJ and/or MMEJ to be evaluated—for example in the context of drug screening and/or in the context of evaluating a given drug in a clinical trial.

For example, a phase I clinical trial of an ATM inhibitor (AZD1390) in combination with radiation for glioblastomas is performed. PBMCs are collected before, and one week after, initiation of the AZD1390 treatment, and the methods and compositions described herein are used to evaluate DSBR by HR, NHEJ and/or MMEJ in these PMBCs. To provide additional controls, the PBMCs collected before treatment may be split into multiple batches, some left untreated, some treated with NU7441, and some treated with AZD1390 directly (i.e. ex vivo).

In another example, a phase II trial enrolls patients with triple negative breast cancer or known BRCA1 or BRCA2 mutations and is designed to evaluate whether concurrent cisplatin and radiation provides an improved palliative benefit for patients with metastatic or recurrent disease. Core biopsies are obtained from each patient. The biopsy samples are dissociated to produce cell aggregates and short-term cultures are established. Nucleofection (or any other suitable transfection method) is used to transfect the dissociated cells with Cas9, gRNA, and donor template, as described above, and then the various compositions and methods described herein are used to evaluate DSBR occurring by HR, NHEJ and MMEJ in the cells derived from the biopsy samples.

In another example, personalized care of cancer patients is achieved by obtaining a sample of tumor cells from a patient, and then performing a method as described herein to evaluate DSBR occurring by HR, NHEJ and MMEJ in the patient's tumor cells, and then selecting a treatment regimen for that patient based on the specific DSBR pathways that are active or defective in the patient's cancer cells.

### Example 3

### Additional Experimental Details

This example describes certain additional details, additional materials and methods, and additional results relating to the identification and analysis of pathway-specific DSBR signatures described above in Example 1 and 2.

Genome editing technologies (TALEN, Zinc Fingers, CRISPR/Cas9) use directed endonucleases to create a double strand break (DSB). Because the objective of editing is a knock-in genomic change, previous investigators have used various cell cycle perturbations(26, 27) or DNA-PKcs inhibitors(28) to bias the repair outcome towards homologous recombination over end-joining. For example, synchronizing a cell population such that editing takes place during S/G2, increases homology directed repair by 2-3 fold. The use of both NHEJ and MMEJ after a Cas9 break is indicated by the frequent presence of microhomology at breaksites(29), which are increased in the absence of key NHEJ factors or with DNA-PKcs inhibition(30, 31). Thus, it is well established that all three HR/NHEJ/MMEJ pathways are utilized in response to double strand breaks created by CRISPR/Cas9. We elected to use Cas9 in the methods described herein, rather than other genome editing enzymes, because Cas9 creates a blunt ended break, most similar to physiologic breaks or radiation-induced breaks, and the breaks occur at a defined location. However, other enzymes that create DSBs, and preferably blunt ended DSBs could be used. In our methods the specific analysis of repair profiles in defined controls enables distillation of the most informative profiles (DSBR signatures) associated with each DSBR pathway. We utilized the characteristic reads/DSBR signatures that we identified as quantitative endpoints in sequence analysis (which we refer to herein as DSBR-seq) or to generate primer and probe sets to selectively detect and measure those characteristic reads/DSBR signatures by droplet digital PCR (which we refer to herein as DSBR-ddPCR).

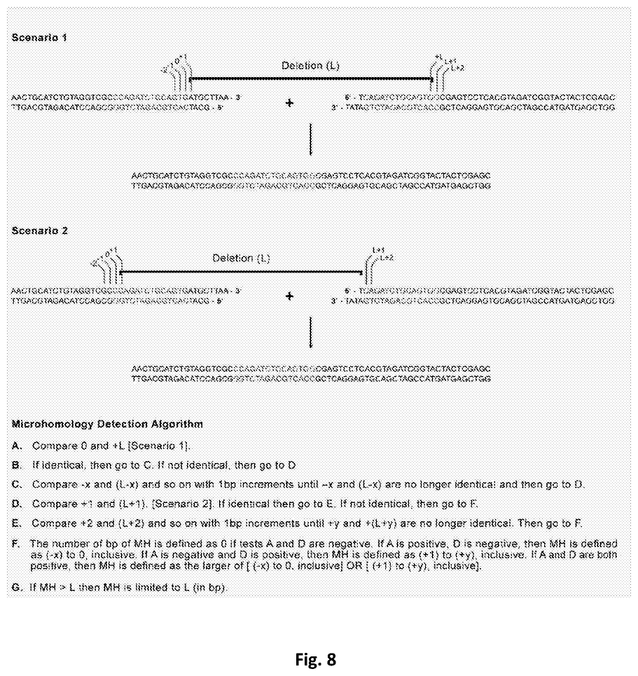

After a DSB is created, the most rapid and most commonly used DSBR pathway used is NHEJ (FIG. 7 (prior FIG. 2). More than 50% of all breaks created by ionizing radiation, for instance, are repaired within one hour by NHEJ(32). The double strand break end is recognized and bound by the abundant Ku heterodimer composed of Ku70(XRCCS) and Ku80 (XRCC6). The complex binds the DNA-PKcs kinase, leading to autophoshorylation, and recruitment of end-processing nucleases, such as Artemis. Finally, the DNA Ligase IV complex, containing LIG4 and XRCC4, completes ligation of the two ends. A form of “backup” or “alterative” endjoining was discovered in eukaryotes, from yeast(33, 34) to human(35, 36), through the observation that end-joining can still occur in the absence of XRCC5/6 or DNA-PKcs. While LIG4 and XRCC4 loss is embryonically lethal, XRCC5/6 or DNA-PKcs null mice are viable. As end-joining is physiologically used during V(D)J recombination in B and T cell maturation, the V(D)J breakpoints formed in absence of XRCC5/6 in knockout mice were found to involve larger deletions and frequent use of stretches of microhomology(37). Since these observations were made in NHEJ-deficient models, the pathway has been more clearly defined to involve end-resection through Mre11 and CTIP to expose single stranded 3′ ends, followed by annealing of microhomologies and synthesis by POLQ(38-40), flap cleavage by XPF-ERCC1, and finally ligation via LIG3 or LIG1. Thus, the mechanistic distinctions between NHEJ and MMEJ include the use of end-resection, the use of POLQ for annealing and synthesis steps, and different ligases used to complete repair. Though both NHEJ and MMEJ are ultimately end-joining based mechanisms using a ligase to complete repair, MMEJ has more factors in common with HR since the first step of both pathways involves end-resection. In developing DSBR-seq, we have used cells deficient in LIG4 and have used the DNA-PKcs inhibitor NU7441. For MMEJ, we have used POLQ deficient cells and the cdk4,6 inhibitor palbociclib to inhibit resection.

Since the initial discoveries were in NHEJ-deficient hosts, alternative end-joining (Alt-EJ) or MMEJ was characterized as a backup pathway. However, in NHEJ competent cells, MMEJ has a physiologic role in some circumstances as more than just a backup pathway, such as in the response to DNA damaging agents. Whole genome sequencing of radiation-induced cancers provides an insight into the mechanism of repair after therapeutic radiation. In these cancers, the distinguishing feature is the predominance of deletions (2-100 bp) in size with microhomology, reflecting the use of MMEJ.(23)

Since a homologous sister chromatid is only available in S and G2 phases of the cell cycle, HR is tightly cell cycle regulated. The initial resection step is mediated through CTIP and Mre11, and CTIP is only active following phosphorylation by CDK2 at the G1-S transition. Exposed single stranded 3′ ends are coated with the single stranded DNA binding RPA complex, which inhibits MMEJ. The RPA coated strand is transformed into a Rad51 coated filament through the mediator functions of BRCA1, PALB2, and BRCA2. The RAD51 filament invades into complementary DNA and initiates synthesis of DNA and formation of complex Holliday junctions that repair the defect. In HR defective cancers, there is an overrepresentation of small deletions with microhomology at the breakpoints(13), indicating that MMEJ acts as a backup pathway to HR and POLQ loss is synthetically lethal with other HR factors, such as FANCD2(40). In developing DSBR-seq, we have used BRCA1 deficient cells and have inhibited both MMEJ and HR through limiting resection via the cdk4,6 inhibitor palbociclib.

The AAVS1 genomic location was chosen due to its characterization as a safe site of integration without any known phenotypic effects. Transfection of the pX330 plasmid, containing the Cas9 and gRNA expression cassettes, is performed by nucleofection or liposome-based techniques. To detect DNA repair by homologous recombination (HR), a donor template is used containing three substitutions, one on the gRNA side of the break and two on the other side of the break, destroying the protospacer adjacent motif (PAM) and thereby limiting cut-recut cycles. A 500 bp amplicon surrounding the break is used for rapid measurement of DSBR errors via the widely used T7 endonuclease I assay. A 201 bp amplicon surrounding the cut site, but outside of the HR template is amplified, purified via E-gel electrophoresis system, converted into Illumina sequencing libraries, quantified via qPCR, and then sequenced at 200,000× read depth. Paired end 150 bp (PE150) reads are obtained, affording a central 100 bp region of high sequence fidelity adjacent to the cut site. The PE150 reads are merged using PEAR and then condensed into a set of unique reads. Unique reads that only occur once are discarded, as they are likely low-quality reads due to sequencing error. These unique, nonsingleton reads are aligned to the reference sequence using pairwise alignment through Python. The indels and substitutions are cataloged relative to the breaksite.

We developed a custom algorithm to detect microhomology which we applied to each unique read (FIG. 8). As CRISPR/Cas9 technology creates a double strand break at a defined location, we analyzed the sequence upstream of the deletion and compared it to the sequence deleted and also ran the analysis in the opposite orientation, comparing sequence downstream of the deletion with the deleted sequence. The algorithm was validated by evaluating the breakpoints of short deletions in BRCA1 and BRCA2 deficient breast and ovarian cancers in The Cancer Genome Atlas project, which exhibited an increase in microhomology compared to BRCA1/2 wild type tumors (36.6 vs. 28.1%, p<0.0001).

Clearly distinguishing NHEJ and MMEJ on the basis of size of the deletion and the extent of microhomology used is challenging for three reasons. First, a small stretch of microhomology (1-2 bp) can happen frequently at a deletion by chance. Second, there is debate as to whether NHEJ can also utilize 1-2 bp of microhomology(41). Third, in biochemical studies, it appears only 1 bp of microhomology is strictly required for POLQ-mediated annealing of 3′ ends(42). Thus, the definition of a MMEJ genomic scar, gleaned from a variety of sources, is often cited as deletions greater than 5bp and either >2 or >3bp of microhomology. We employed these criteria as a starting point for initial assessment of DSBR-seq and found that even these non-empirically defined criteria did capture pathway usage in broad terms.

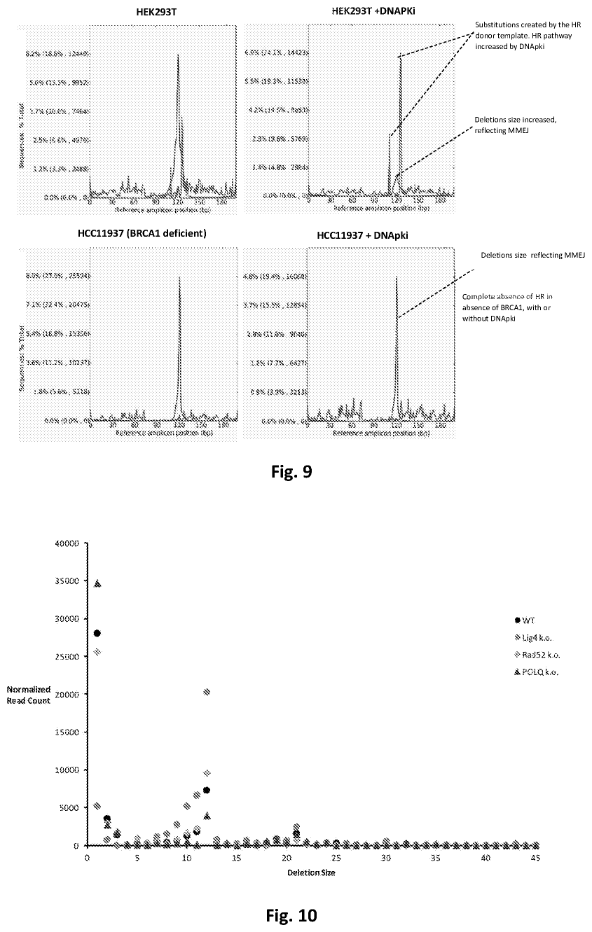

While several previous reports found that Cas9 breaks rely on NHEJ/MMEJ/HR pathways for repair, the specific read profiles dependent on each pathway were not previously apparent. The profile of deletion sizes and extent of microhomology differs between break sites, likely owing to site dependent availability of microhomology. We utilized the DNA-PKcs inhibitor NU7441 to measure NHEJ inhibition. For analysis of HR, we chose the well characterized HCC1937 breast cancer cell line, which has a pathogenic, homozygous BRCA1 5382C insertion mutation. In FIG. 9, examples of the profile of deletions, substitutions, and insertions is shown. The frequency of each alteration at each base pair position within a sequencing amplicon is graphed and since the DSB was created at position 120 in the genomic DNA, the deletion peak is positioned at this base pair. In HEK293T cells, the amplitude of the deletion peak decreases by more than 90% with use of the DNA-PKcs inhibitor NU7441 and the width increases, reflecting use of MMEJ (FIG. 9 top left and right panels). Since our donor template contained three characteristic substitutions at two locations on both sides of the breaksite, HR is represented by two green substitution peaks. These HR peaks increased in frequency as DNApk is inhibited, reflecting transfer of NHEJ events to HR events. In the HCC1937 BRCA1 deficient cell line, no HR is observed, with or without treatment with a DNA-PKcs inhibitor, confirming the measurement of homologous recombination.

To more clearly differentiate between MMEJ and NHEJ in specific reads, we utilized the CML cell line HAP1 and isogenic LIG4 and POLQ deleted HAP1 cell lines created through CRISPR/Cas9 editing. In the absence of LIG4, small deletions, particular 1 bp reads are highly suppressed (FIG. 10) whereas these reads are enhanced in the POLQ deleted line or unaffected by loss of RAD52, the key factor in a rarely used pathway, single strand annealing. Conversely, deletions larger than 5 bp are enhanced in absence of LIG4, but suppressed by POLQ loss. Thus, at this breaksite, a 1 bp deletion is most common, reflecting NHEJ, and a 12 bp deletion is the second most common size, reflecting MMEJ. We evaluated 10 other cell lines, finding an identical shape to the deletion curve with 1 and 12 bp deletions as the most common whereas the relative usage differed amongst them (FIG. 10B). These data support the universality of the read profiles across all cell lines and the utility in measuring NHEJ and MMEJ.

Further, we found the repair profile changes in the LIG4 deficient HAP1 background are similar to those found with DNA-PKcs inhibition in 293T cells at a sequence level (FIG. 1). The top 20 most common non-WT reads are displayed in heat map format. A specific 1 bp deletion is most common, but suppressed by DNA-PKcs inhibition or by LIG4 loss. Conversely, a 12 base pair deletion that displays 5 bp of microhomology is the second most common deletion. It is enhanced by the DNA-PKcs inhibitor in both HAP1 and 293T cells or by LIG4 deficiency and suppressed by POLQ deficiency.

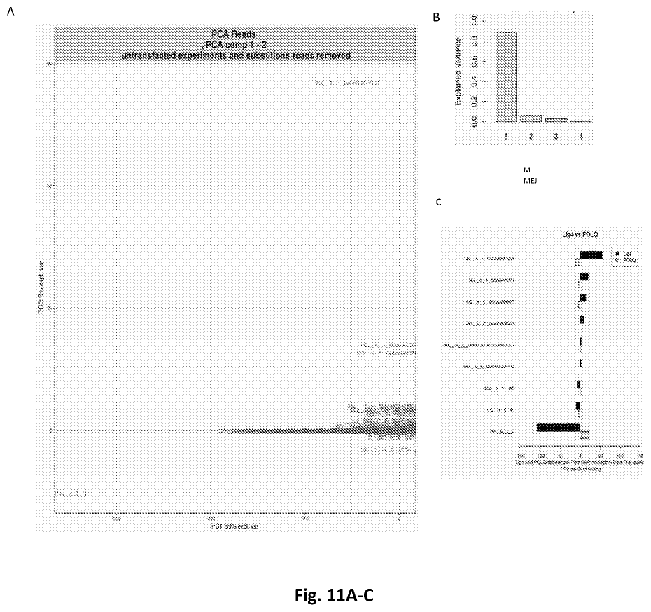

When evaluating all three pathways, using the literature-derived criteria for MMEJ vs. NHEJ, we found 90% suppression of NHEJ, a nearly 2-fold increase in MMEJ, and a 2.8 fold increase in HR in LIG4 deficient cells (FIG. 111). A similar effect was seen with DNApkcs inhibition (FIG. 11). POLQ deficient cells exhibited suppressed MMEJ and increased HR, but interestingly no change in NHEJ. As MMEJ and HR share an upstream resection step, we propose that an already resected 3′ end destined for MMEJ cannot be resolved by NHEJ, which does not utilize resection. This is an example of the type of observation uniquely enabled by our system with its ability to detect three different DSBR mechanisms simultaneously at a single locus. A 50% reduction in MMEJ was also seen using the EJ2-GFP reporter construct when POLQ is knocked down with siRNA(40), potentially indicating there are other mechanisms besides POLQ that can mediate MMEJ.

To statistically define the repair reads most reflective of each pathway, we used principal component analyses (PCA) using the unique, nonsingleton reads and their normalized frequencies (FIG. 11). Four principal components (PCs) were statistically significant (p<0.05). PC1 clearly represented NHEJ, defining the cluster of experiments in the LIG4 deficient cell line. A one base pair deletion, was both the most frequent indel overall and also the most highly suppressed in the absence of LIG4. The dominance of the NHEJ reads through all experiments is consistent with the estimation that 85% of DSBs are repaired through NHEJ. PC2 represents MMEJ as the 12 bp read with 5 bp of microhomology (−12del/5 mm) was the most characteristically different read in the absence of POLQ, though two other 10 and 11 bp dels with 2 bp of microhomology were also contributors.

The effects of LIG4 deficiency may be more pronounced in these experiments than the reciprocal effect of POLQ, deficiency because HR is the primary rescuing pathway in absence of POLQ, rather than NHEJ (FIG. 1).

The results of this analysis have several important implications. First, as just a few reads were highly characteristic between pathways, these results demonstrate that there is a high degree of overlap between scars left behind by both pathways. Thus, validation of the repair signatures with isogenic controls was critically important.

Second, since the highly characteristic reads were also the most common reads, the DSBR-seq analysis can be adapted away from using non-empiric, literature-based definitions of MMEJ and NHEJ scars in favor of simply analyzing these empirically derived reads. Using these empirically derived reads, we observed identical results as in FIG. 1G-I. Thus, now that we have discovered these read/DSBR signatures based on our extensive bioinformatic analysis, for future DSBR-seq studies the bioinformatic needs will be greatly reduced. In particular at this locus, there would be no need to employ a microhomology search algorithm in the future (see FIG. 8). Instead sequence alignment and quantification of the characteristic reads is all that would be required.

Third, we surprisingly found two other principle components (PCs), reflecting unknown subpathways. PC3 clustered small deletions of 2-3 bp in size, which we suspect reflect the end-processing events that can occur with NHEJ. Thus, our approach may enable analysis of DSBR subpathways using additional isogenic knockout control cell lines.

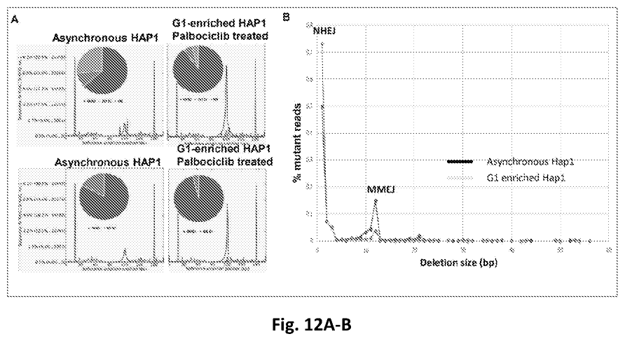

Cell cycle effects on NHEJ/MMEJ/HR frequency. HR is limited to S/G2 phases of the cell cycle through CDK2 regulation of a mediator of end-resection (CTIP).(43, 44) While there are conflicting reports as to the usage of MMEJ throughout the cell cycle, we have found that MMEJ is also highly dependent on cell cycle. When applying the cdk4,6 inhibitor palbociclib, cells are arrested in G1. The frequency of introduced errors overall increases ˜4 fold, but the proportion of -12de1/5mm reads is highly suppressed and the 1 bp del is increased (FIG. 12). Thus, while the overall “error-prone” NHEJ is increased, MMEJ is suppressed. HR is also suppressed as expected. These results underline the power of our technique to assess all changes in DSBR pathway choice simultaneously. A weakness of reporter cassettes is that any perturbation of cell cycle can also lead to changes in the readout as end resection is restricted to S and G2 phases of the cell cycle. Since we have found both HR and MMEJ to be cell cycle dependent, repair defects can be separated from a cell cycle effect if HR and MMEJ are differentially affected.

### Example 4

### Proof-of-Concept for Utility of DSBRseq in Studying Biological Questions

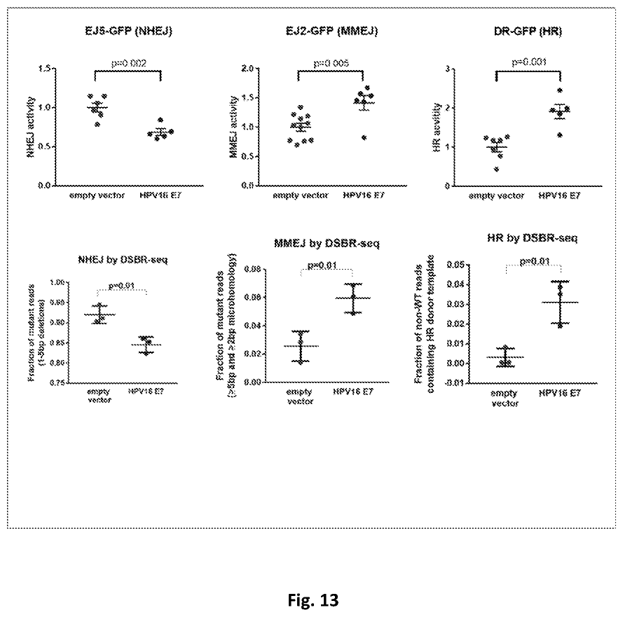

Cancers associated with the human papillomavirus are well known clinically to respond well to DNA damaging agents(45) and the viral oncogene E7 is known to inhibit DSBR(46). The specific pathway which is affected by DSBR is unknown, though indirectly other reports have suggested sensitivity to PARP inhibitors as suggestive of an HR defect. We tested all viral oncogenes in the DRGFP, EJ5GFP, and EJ2GFP cassettes and found suppression of NHEJ and enhancement of MMEJ and HR (FIG. 13). These results support that the mechanism of radiosensitivity in HPV associated oropharyngeal cancer is the inhibition of NHEJ. While the experiments using cassette reporters were informative, the time required to complete them was 4 months. We replicated these findings using 3 DSBR-seq experiments over 2 weeks—underscoring the power of our new methods.

### Example 5

### Determining NHEJ, MMEJ, and HR Signatures at Multiple Genomic Locations

The work described in Examples 1 and 2 above extensively characterized HR/NHEJ/MMEJ DSBR pathway signatures at one locus (AAVS1). Similar studies at additional loci will validate the approach, and provide opportunities for multiplexing DSBR-seq and DSBR-ddPCR.

The existing AAVS1 site is located within a euchromatic site. Two additional sites are selected within euchromatic sites and three are selected within facultative heterochromatic sites. Using multiple DSB sites in differing chromatin states will broaden the applicability of DSBR-seq.

When DSBs are created by ionizing radiation, most are repaired rapidly within 4 hours using NHEJ whereas a smaller proportion of breaks are repaired with delayed kinetics, resolving between 6-24 hours. The “slow” and “fast” phases of DSBs can be observed using cytogenetics(32), H2AX foci resolution kinetics(47), and can also be seen following a DSBs created by Cas9(48). The slow phase of DSBR repair is proposed to be reflective of either complex breaks that require HR to repair or breaks embedded within compacted heterochromatin which block access to NHEJ factors.

Following a DSB, ATM phosphorylates KAP1 which is closely associated with heterochromatin proteins that limit movement of nucleosomes, notably HP1(49). pKAP1 fails to bind HP1, allowing for chromatin relaxation and access for DNA repair factors(49, 50). The process of DSB generation through CRISPR/Cas9 may alter the native chromatin state directly surrounding the break(51) and second, the native heterochromatin can decrease the efficiency of Cas9 editing(52, 53). Thus, the pathway choices used at heterochromatic breaks may differ from euchromatin, either due to the inherent biology of DSBR within differing chromatin states or because of changes in the state of chromatin induced by Cas9. Of note, the chromatin state of integrated reporter cassettes generally is not known or considered. Studies are performed at 2 additional euchromatin loci and 3 heterochromatic loci. Regions of facultative rather than constitutive heterochromatin will be selected as facultative regions are far more prevalent and therefore broader in applicability to DSBR studies.

A second purpose of including additional loci is to amplify the signal available within one experiment. Multiple gRNAs can be co-transfected with Cas9, inducing breaks at several locations and the DSBR-seq and DSBR-ddPCR methods described herein can measure repair results simultaneously. In some cell lines, the MMEJ signal can be lower than 5% of all mutant reads and thus additional signal will improve reproducibility.

TABLE 2
Controls for use in assessing 6 total genomic loci.

| DSBR Pathway | Positive Control Cells | Negative Control Cells |
|---|---|---|
| NHEJ | HAP1 cells | LIG4 deficient HAP1 |
| | 293T cells | 293T cells, NU7441 |
| | U2OS | U2OS, NU7441 |
| MMEJ | HAP1 cells | POLQ deficient HAP1 |
| | HAP1 cells | HAP1, palbociclib |
| | 293T cells | 293T, palbociclib |
| | U2OS | U2OS, palbociclib |
| HR | HCC1937 | HCC1937 (BRCA1 |
| | complemented with | deficient) |
| | functional BRCA1 | |
| | 293T cells | 293T cells, palbociclib |
| | U2OS | U2OS, palbociclib |

Experimental Design: The HAP1, 293T, U2OS, and HCC1937 cell lines are used, building upon existing data. The chromatin state of selected loci is defined on the basis of minimal sensitivity to DNase I and the presence of H3K27me3 marks in the K562 cell line by ChlPseq in the ENCODE Project. The HAP1 cell line is a derivative of K562, a “Tier 1” characterized cell line by ENCODE and thus these data are readily available. 293T cells were included within Tier 3, but neither DNAse I nor H3K27me3 data are available at this time. U2OS and HCC1937 were not included in ENCODE. Thus, genomic loci are selected on the basis of K562 and the same sites are used in all cell lines as the purpose of including differing sites is to improve applicability to general DSBR studies rather than study chromatin biology explicitly.

In all cell lines, the pX330 plasmid is used, containing Cas9 and gRNA expression cassettes. Donor template DNA is co-transfected with characteristic substitutions as described. The repair profiles and pathway dependence for the five new genomic loci are characterized as previously performed, including principal component analyses and assignment to MMEJ and NHEJ using the HAP1 WT, HAP1 LIG4 deficient, and HAP1 POLQ deficient cell lines. Experiments using the DNA-PKcs inhibitor NU7441 and the cdk4,6 inhibitor palbociclib in 293T and U2OS cells are used to validate changes in reads assigned to each pathway. The final DSBR-seq MMEJ and NHEJ endpoints are the fraction of all mutant reads corresponding to each pathway.

Finally, both single locus and multilocus DSBR-seq in the U2OS cell line is directly compared against the DR-GFP, EJ2-GFP, and EJ5-GFP reporter systems which are integrated into the U2OS background. NU7441 and palbociclib have demonstrated a large dynamic range at the AAVS1 locus for all three pathways and thus these agents are used in U2OS for both DSBR-seq and the reporter cassettes using identical conditions.

Statistical Definitions: Dynamic range is defined as the percentage of mutant reads corresponding to NHEJ, MMEJ, and HR in the positive controls (wild type cells) divided by the percentage of mutant reads in the negative controls (pathway deficient cells). The coefficients of variation are based on repeated, biologically independent replicates in positive and negative controls. Finally, the Z-factor statistic provides an integrated reflection of assay strength, incorporating both dynamic range and standard deviations of positive and negative control samples.

Outcomes: Identifiable reads that are highly characteristic of NHEJ and MMEJ at each locus are identified. LIG4 deficiency and NU7441 may suppress the same reads that are assigned to NHEJ and conversely, POLQ deficiency or palbociclib may increase the frequency of those reads. LIG4 deficiency and NU7441 may increase the frequency of reads assigned to MMEJ, which may be conversely suppressed by POLQ deficiency and treatment with palbociclib. Single-locus DSBR-seq is expecte to have equivalent dynamic range, coefficients of variation, and Z-statistic to the reporter constructs using the positive and negative controls outlined in Table 4 above. Multilocus DSBR-seq can provide a superior dynamic range and therefore an improved Z-statistic for all three reporters.

### Example 6

### Validation of DSBR-ddPCR as a rapid tool for use in cancer research

Using existing data and the newly characterized loci obtained described above, the specific NHEJ, MMEJ, and HR-specific signature are detected using droplet digital PCR (ddPCR). Droplet digital PCR machines (Biorad QX200, Raindance RainDropPlus) or digital PCR machines (ThermoFisher QuantStudio 3D) are widely available at most research institutions. ddPCR has emerged as an important technology in the quantification of cancer-derived single nucleotide variants in cell free DNA. Thus, it is well established that ddPCR can detect small, even single nucleotide changes in DNA samples. The present invention teaches the creation and use of dedicated ddPCR primer/probe sets that are specific to NHEJ, MMEJ, and HR generated alleles.

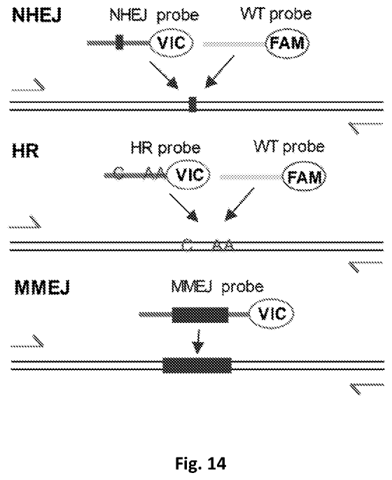

Experimental Design. Genomic DNA is extracted from cells. The genomic DNA is digested using frequently cutting endonucleases, resulting in ˜1 kb fragments. Enzymes are selected that do not cut within chosen amplicons. At the AAVS1 breaksite, there are two types of reads (−1del and −12del/5 mm) that provide most of the contribution to the NHEJ and MMEJ principal components. For NHEJ, a 1 bp deletion can be captured using droplet digital PCR with primers surrounding the breaksite and probe (FAM labeled) specific to the deletion (FIG. 14). A competitive probe matching wild type sequence is HEX labeled. Genomic DNA is digested with randomly cutting endonucleases and then droplet digital PCR (Biorad QX200) partitions the sample allowing PCR reactions in droplets with single molecule templates. The ratio of FAM to HEX labeled droplets represent the percentage of cells containing a NHEJ repair event. Similarly, the HR read, containing three characteristic substitutions, is captured with a probe specific to the edited read and a competitor probe. For the −12del/5 mm deletion, the large difference between the wild type and mutant read allows for just one probe matching the read without a competitor probe. A reference amplicon in another location is used and the ratio of −12del/5 mm positive droplets to the reference amplicon droplets represents the percentage of cells with MMEJ reads.

NHEJ, MMEJ, and HR-specific primer/probe sets are also developed to 5 additional loci. Characterization of the sensitivity and specificity of ddPCR is performed using plasmids containing mutant sequences which are serially diluted either in water or in wild type genomic DNA.

Using the same positive and negative controls, dynamic ranges, coefficients of variation, and Z-statistics are determined for single locus DSBR-ddPCR. From one experimental condition from a 10 cm dish, generally several μg of DNA is obtained—which is enough material for multiple DSBR-ddPCR experiments as only 100 ng are used per reaction. The Biorad QX200 machine includes a 96 well plate format and thus a typical array of experiments from one condition involves three technical replicates each for the three pathway assays, and positive and negative amplicon controls.

In addition to determining the statistical performance of each locus separately, tests are also performed to determine whether combining results from all 6 loci can improve performance, particularly the dynamic range of the MMEJ reads, which are less frequent than HR and NHEJ reads.

Statistical Definitions The number of NHEJ or HR droplets divided by the corresponding number of WT droplets provides the absolute fraction of reads containing NHEJ and HR reads. The number of MMEJ droplets divided by a reference amplicon (FAM) corresponds to the fraction of MMEJ reads. Dynamic range, coefficients of variation, and the Z-statistic are determined using the identical positive and negative controls.

Outcomes: NHEJ, MMEJ, and HR amplicons are expected to be highly specific with less than 5 positive droplets per reaction when using genomic DNA from cells untransfected by Cas9. Dilution series are used to demonstrate sensitivity to near single molecule levels, as has been observed with other ddPCR applications.

Numbers in parentheses or in superscript in these Examples refer to the numbered references in the reference lists that follows this Examples section.

### Reference List for Examples 1 & 2

1. Ma et al. Genomic analysis of exceptional responders to radiotherapy reveals somatic mutations in ATM. Oncotarget. 2017; 8(6):10312-23.

2. Stover et al. Biomarkers of Response and Resistance to DNA Repair Targeted Therapies. Clin Cancer Res. 2016; 22(23):5651-60.

3. Robson et al. Olaparib for Metastatic Breast Cancer in Patients with a Germline BRCA Mutation. N Engl J Med. 2017; 377(6):523-33.

4. Kaufman et al. Olaparib monotherapy in patients with advanced cancer and a germline BRCA1/2 mutation. J Clin Oncol. 2015; 33(3):244-50.

5. Ledermann et al. Olaparib maintenance therapy in platinum-sensitive relapsed ovarian cancer. N Engl J Med. 2012; 366(15):1382-92.

6. Ledermann et al. Olaparib maintenance therapy in patients with platinum-sensitive relapsed serous ovarian cancer: a preplanned retrospective analysis of outcomes by BRCA status in a randomised phase 2 trial. Lancet Oncol. 2014; 15(8):852-61.

7. Mirza et al. Niraparib Maintenance Therapy in Platinum-Sensitive, Recurrent Ovarian Cancer. N Engl J Med. 2016; 375(22):2154-64.

8. Mateo et al. DNA-Repair Defects and Olaparib in Metastatic Prostate Cancer. N Engl J Med. 2015; 373(18):1697-708.

9. Mutter et al. Bi-allelic alterations in DNA repair genes underpin homologous recombination DNA repair defects in breast cancer. J Pathol. 2017; 242(2):165-77.

10. Popova et al. Ploidy and large-scale genomic instability consistently identify basal-like breast carcinomas with BRCA1/2 inactivation. Cancer Res. 2012; 72(21):5454-62.

11.Telli et al. Homologous Recombination Deficiency (HRD) Score Predicts Response to Platinum-Containing Neoadjuvant Chemotherapy in Patients with Triple-Negative Breast Cancer. Clin Cancer Res. 2016; 22(15):3764-73

12. Timms et al. Association of BRCA1/2 defects with genomic scores predictive of DNA damage repair deficiency among breast cancer subtypes. Breast Cancer Res. 2014; 16(6):475.

13.Davies et al. HRDetect is a predictor of BRCA1 and BRCA2 deficiency based on mutational signatures. Nat Med. 2017; 23(4):517-25.

14.Johnson et al. Stabilization of mutant BRCA1 protein confers PARP inhibitor and platinum resistance. Proc Natl Acad Sci U S A. 2013; 110(42):17041-6.

15. Jaspers et al. Loss of 53BP1 causes PARP inhibitor resistance in Brca1-mutated mouse mammary tumors. Cancer Discov. 2013; 3(1):68-81.

16. Pettitt et al. Genome-wide and high-density CRISPR-Cas9 screens identify point mutations in PARP1 causing PARP inhibitor resistance. Nat Commun. 2018; 9(1):1849.

17.Hu et al Quantitative Chemotherapy Genetic Interaction Map Reveals Factors Associated with PARP Inhibitor Resistance. Cell Rep. 2018; 23(3):918-29.

18.Polkinghorn et al. Androgen receptor signaling regulates DNA repair in prostate cancers. Cancer Discov. 2013; 3(11):1245-53.

19. Squatrito et al. 53BP1 is a haploinsufficient tumor suppressor and protects cells from radiation response in glioma. Cancer Res. 2012; 72(20):5250-60.

20. Muvarak et al. c-MYC Generates Repair Errors via Increased Transcription of Alternative-NHEJ Factors, LIG3 and PARP1, in Tyrosine Kinase-Activated Leukemias. Mol Cancer Res. 2015; 13(4):699-712.

21.Bentley et al. DNA double strand break repair in human bladder cancer is error prone and involves microhomology-associated end-joining. Nucleic Acids Res. 2004; 32(17):5249-59.

22.McCormick et al. Ovarian Cancers Harbor Defects in Nonhomologous End Joining Resulting in Resistance to Rucaparib. Clin Cancer Res. 2017; 23(8):2050-60.

23.Behjati et al. Mutational signatures of ionizing radiation in second malignancies. Nat Commun. 2016; 7:12605.

24. Weinstock et al. Assaying double-strand break repair pathway choice in mammalian cells using a targeted endonuclease or the RAG recombinase. Methods Enzymol. 2006; 409:524-40.

25.Gunn et al. I-Seel-based assays to examine distinct repair outcomes of mammalian chromosomal double strand breaks. Methods Mol Biol. 2012; 920:379-91.

26.Feng et al. Rad52 inactivation is synthetically lethal with BRCA2 deficiency, Proceedings of the National Academy of Sciences of the United States of America, 108 (2011) 686-691.

27.Perez et al. Establishment of HIV-1 resistance in CD4+ T cells by genome editing using zinc-finger nucleases. Nat Biotechnol. 2008; 26(7):808-16.

28. Tebas et al. Gene editing of CCRS in autologous CD4 T cells of persons infected with HIV. N Engl J Med. 2014; 370(10):901-10.

29. Genovese et al. Targeted genome editing in human repopulating haematopoietic stem cells. Nature. 2014; 510(7504):235-40.

30. Hanna et al. Labeling of Breast Cancer Patient-derived Xenografts with Traceable Reporters for Tumor Growth and Metastasis Studies. J Vis Exp. 2016 (117).

31. DeRose et al. Patient-derived models of human breast cancer: protocols for in vitro and in vivo applications in tumor biology and translational medicine. Curr Protoc Pharmacol. 2013;Chapter 14:Unit14 23.

32. Cruz et al. RAD51 foci as a functional biomarker of homologous recombination repair and PARP inhibitor resistance in germline BRCA-mutated breast cancer. Ann Oncol. 2018;29(5):1203-10.

33. Jacobsen et al. Nucleofection: a new method for cutaneous gene transfer? J Biomed Biotechnol. 2006; 2006(5):26060.

34. Vangipuram et al. Skin punch biopsy explant culture for derivation of primary human fibroblasts. J Vis Exp. 2013 (77):e3779.

35. Zare et al. Isolation, cultivation and transfection of human keratinocytes. Cell Biol Int. 2014; 38(4):444-51.

36. Zaragosi L E, Billon N, Ailhaud G, Dani C. Nucleofection is a valuable transfection method for transient and stable transgene expression in adipose tissue-derived stem cells. Stem Cells. 2007; 25(3):790-7.

37. Indumathi et al. Lineage depletion of stromal vascular fractions isolated from human adipose tissue: a novel approach towards cell enrichment technology. Cytotechnology. 2014; 66(2):219-28.

### Reference List for Examples 3, 4, 5, & 6

References 1-25 cited in Examples 3-6 are the same as references 1-25 from Examples 1-2 (see Reference List or Examples 1 & 2 above).

26. Lin/Enhanced homology-directed human genome engineering by controlled timing of CRISPR/Cas9 delivery. Elife. 2014; 3:e04766.

27. Yang. Enrichment of G2/M cell cycle phase in human pluripotent stem cells enhances HDR-mediated gene repair with customizable endonucleases. Sci Rep. 2016; 6:21264.

28. Robert. Pharmacological inhibition of DNA-PK stimulates Cas9-mediated genome editing. Genome Med. 2015; 7:93.

29. van Schendel. Polymerase Theta is a key driver of genome evolution and of CRISPR/Cas9-mediated mutagenesis. Nat Commun. 2015; 6:7394.

30. Schimmel. Mutational signatures of non-homologous and polymerase theta-mediated end joining in embryonic stem cells. EMBO J. 2017; 36(24):3634-49.

31. van Overbeek. DNA Repair Profiling Reveals Nonrandom Outcomes at Cas9-Mediated Breaks. Mol Cell. 2016; 63(4):633-46.

32. Cornforth. X-ray—induced breakage and rejoining of human interphase chromosomes. Science. 1983; 222(4628):1141-3.

33. Boulton. Identification of a Saccharomyces cerevisiae Ku80 homologue: roles in DNA double strand break rejoining and in telomeric maintenance. Nucleic Acids Res. 1996; 24(23):4639-48.

34. Boulton. Saccharomyces cerevisiae Ku70 potentiates illegitimate DNA double-strand break repair and serves as a barrier to error-prone DNA repair pathways. EMBO J. 1996; 15(18):5093-103.

35. Ferguson. The nonhomologous end joining pathway of DNA repair is required for genomic stability and the suppression of translocations. Proc Natl Acad Sci U S A. 2000; 97(12):6630-3

36. Kabotyanski. Double-strand break repair in Ku86- and XRCC4-deficient cells. Nucleic Acids Res. 1998; 26(23):5333-42.

37. Boboila. Alternative end joining catalyzes class switch recombination in the absence of both Ku70 and DNA ligase 4. J Exp Med. 2010; 207(2):417-27.

38. Yousefzadeh. Mechanism of suppression of chromosomal instability by DNA polymerase POLQ. PLoS Genet. 2014; 10(10):e1004654.

39. Wyatt. Essential Roles for Polymerase theta-Mediated End Joining in the Repair of Chromosome Breaks. Mol Cell. 2016; 63(4):662-73.

40. Ceccaldi. Homologous-recombination-deficient tumours are dependent on Poltheta-mediated repair. Nature. 2015; 518(7538):258-62.

41. Pannunzio Non-homologous end joining often uses microhomology: implications for alternative end joining DNA Repair (Amst). 2014; 17:74-80.

42. Kent. Mechanism of microhomology-mediated end joining promoted by human DNA polymerase theta. Nat Struct Mol Biol. 2015; 22(3):230-7.

43. Buis. Mrell regulates CtIP-dependent double-strand break repair by interaction with CDK2. Nat Struct Mol Biol. 2012; 19(2):246-52.

44. Orthwein. A mechanism for the suppression of homologous recombination in G1 cells. Nature. 2015; 528(7582):422-6.

45. Ang. Human papillomavirus and survival of patients with oropharyngeal cancer. N Engl J Med. 2010; 363(1):24-35.

46. Cleary. Biological Features of Human Papillomavirus-related Head and Neck Cancers Contributing to Improved Response. Clin Oncol (R Coll Radiol). 2016; 28(7):467-74.

47. Sharma. High throughput measurement of gammaH2AX DSB repair kinetics in a healthy human population. PLoS One. 2015; 10(3):e0121083.

48. Rose. Rapidly inducible Cas9 and DSB-ddPCR to probe editing kinetics. Nat Methods. 2017; 14(9):891-6.

49. White. The ATM substrate KAP1 controls DNA repair in heterochromatin: regulation by HP1 proteins and serine 473/824 phosphorylation. Mol Cancer Res. 2012; 10(3):401-14.

50. Goodarzi. ATM signaling facilitates repair of DNA double-strand breaks associated with heterochromatin. Mol Cell. 2008; 31(2):167-77.

51. Barkal. Cas9 Functionally Opens Chromatin. PLoS One. 2016; 11(3):e0152683.

52. Daer. The Impact of Chromatin Dynamics on Cas9-Mediated Genome Editing in Human Cells. ACS Synth Biol. 2017; 6(3):428-38.

53. Chen/The Chromatin Structure Differentially Impacts High-Specificity CRISPR-Cas9 Nuclease Strategies. Mol Ther Nucleic Acids. 2017; 8:558-63.

54. Sims. High-throughput RNA interference screening using pooled shRNA libraries and next generation sequencing. Genome Biol. 2011; 12(10):R104.

55. Perez. Establishment of HIV-1 resistance in CD4+ T cells by genome editing using zinc-finger nucleases. Nat Biotechnol. 2008; 26(7):808-16.

56. Tebas. Gene editing of CCRS in autologous CD4 T cells of persons infected with HIV. N Engl J Med. 2014; 370(10):901-10.

57. Genovese. Targeted genome editing in human repopulating haematopoietic stem cells. Nature. 2014; 510(7504):235-40.

58. Hanna J Vis Exp. 2016 (117).

59. DeRose. Curr Protoc Pharmacol. 2013; Chapter 14:Unit14 23.

60. Cruz. RAD51 foci as a functional biomarker of homologous recombination repair and PARP inhibitor resistance in germline BRCA-mutated breast cancer. Ann Oncol. 2018; 29(5):1203-10.

61. Jacobsen. Nucleofection: a new method for cutaneous gene transfer? J Biomed Biotechnol. 2006; 2006(5):26060.

62. Vangipuram. Skin punch biopsy explant culture for derivation of primary human fibroblasts. J Vis Exp. 2013 (77):e3779.

63. Zare. Isolation, cultivation and transfection of human keratinocytes. Cell Biol Int. 2014; 38(4):444-51.

64. Zaragosi. Nucleofection is a valuable transfection method for transient and stable transgene expression in adipose tissue-derived stem cells. Stem Cells. 2007; 25(3):790-7.

65. Indumathi Lineage depletion of stromal vascular fractions isolated from human adipose tissue: a novel approach towards cell enrichment technology. Cytotechnology. 2014; 66(2):219-28.

We claim:

1. A method of identifying double strand break repair (DSBR) sequence signatures in DNA that has undergone repair of a double strand break (DSB), wherein the DSBR sequence signatures are characteristic of, and specific to, the cellular pathway by which the DSB was repaired, the method comprising:
a) contacting the genome of cells of interest and the genome of control cells with an endonuclease to create blunt-ended DSBs at a predetermined location in the genomes of the cells of interest and the control cells, wherein the control cells are deficient in (i) the homologous recombination (HR) DSBR pathway (HR control cells), (ii) the nonhomologous end-joining (NHEJ) DSBR pathway (NHEJ control cells), or (iii) the microhomology-mediated end-joining (MMEJ) repair pathway (MMEJ control cells),
b) determining the DNA sequences of the genomic DNA from the cells of interest and from the control cells in the region spanning the location of the DSBs after the cells have been maintained in culture for sufficient time to allow repair of the DSBs by the cells' innate DNA repair machinery, and
c) comparing the DNA sequences identified in step (b) from the cells of interest to those of the HR control cells, the NHEJ control cells and/or the MMEJ control cells, wherein: (i) sequence signatures that differ between the cells of interest and the HR control cells are HR DSBR signature sequences, (ii) sequence signatures that differ between the cells of interest and the NHEJ control cells are NHEJ DSBR signature sequences, and (iii) sequence signatures that differ between the cells of interest and the MMEJ control cells are MMEJ DSBR signature sequences.

2. The method of claim 1, wherein the endonuclease is a Cas9 endonuclease.

3. The method of claim 1, wherein step a) comprises transfecting the cells of interest and the control cells with a vector comprising a Cas9 endonuclease expression cassette and a guide RNA (gRNA) cassette, wherein the gRNA is specific for the predetermined location in the genome.

4. The method of claim 1, wherein the predetermined location in the genome is within the AAVS1 safe-harbor site on human chromosome 19 (locus PPP1R12C).

5. The method of claim 4, wherein the predetermined location in the genome is between nucleotides 55115755 and 55115754 of human chromosome 19, GRCh38.p12.

6. The method of claim 1, wherein the predetermined location in the genome is within the AAVS1 safe-harbor site on chromosome 19 (locus PPP1R12C) of the human genome and wherein step a) comprises transfecting the cells of interest and the control cells with a vector comprising a Cas9 endonuclease expression cassette and a guide RNA (gRNA) cassette, wherein the gRNA is specific for the predetermined location within the AAVS1 safe harbor site.

7. The method of claim 3 or claim 6, wherein the vector is the pX330 vector.

8. The method of claim 1, wherein step a) further comprises transfecting the cells of interest and the control cells with a donor template for homologous recombination, wherein the donor template comprises one or more mutations that are not present at the predetermined location in the genome of the cells of interest or the genome of the control cells.

9. The method of claim 1, wherein: (i) in the HR control cells a component of the HR DSBR pathway has been knocked out genetically and/or inhibited pharmacologically, (ii) in the NHEJ control cells a component of the NHEJ DSBR pathway has been knocked out genetically and/or inhibited pharmacologically, and/or (iii) in the MMEJ control cells a component of the HR DSBR pathway has been knocked out genetically and/or inhibited pharmacologically.

10. The method of claim 1, wherein step b) comprises determining the DNA sequences of the genomic DNA from the cells of interest and from the control cells using a next generation sequencing (NGS) method.

11. The method of claim 1, wherein step b) comprises determining DNA sequences resulting from more than 1,000 different repair events using a next generation sequencing (NGS) method.

12. The method of claim 1, wherein step b) comprises determining DNA sequences resulting from more than 10,000 different repair events using a next generation sequencing (NGS) method.

13. The method of claim 1, wherein step b) comprises determining DNA sequences resulting from more than 100,000 different repair events using a next generation sequencing (NGS) method.

14. The method of claim 1, wherein step b) comprises determining DNA sequences resulting from more than 200,000 different repair events using a next generation sequencing (NGS) method.

15. The method of claim 1, wherein in step b) the cells have been maintained in culture for about 3 days.

16. The method of claim 1, further comprising culturing the cells of interest and the control cells for sufficient time to allow repair of the DSB by the cells' innate DNA repair machinery prior to performing step b).

17. The method of claim 16, wherein the cells are maintained in culture for about 3 days

18. The method of claim 1, further comprising performing a PCR reaction to generate PCR products that that span the location of the DSBs in the genomic DNA from the cells of interest and from the control cells the prior to performing step b), and, in step b), determining the DNA sequences of the PCR products.

19. The method of any of the preceding claims wherein the cells of interest and/or the control cells are cancer cells.

20. The method of any of the preceding claims wherein the cells of interest and/or the control cells are peripheral blood mononuclear cells (PBMCs).

21. The method of any of the preceding claims wherein the cells of interest and/or the control cells are obtained from a biopsy sample.

22. The method of any of the preceding claims wherein the cells of interest and/or the control cells are obtained from a patient-derived xenograft (PDX).

23. The method of any of the preceding claims wherein the cells of interest and/or the control cells are human cells.

24. The method of any of the preceding claims further comprising quantifying the relative usage of HR, NHEJ and MMEJ repair mechanisms in the cells of interest by quantifying the number of repair events or sequence reads comprising HR DSBR signature sequences, NHEJ DSBR signature sequences, and MMEJ DSBR signature sequences.

25. The method of any of the preceding claims, wherein the method is performed in the presence or absence of an inhibitor or candidate inhibitor of double strand break repair during the time that the DSB is generated and/or during the recovery time after DSB generation when DSBR would normally occur, to monitor the effect of that inhibitor or candidate inhibitor on the DSBR process and/or on the relative usage of the HR, NHEJ, and/or MMEJ pathways of DSBR.

26. A method of assessing the activity and/or usage of homologous recombination (HR), nonhomologous end-joining (NHEJ) and/or microhomology-mediated end-joining (MMEJ) DSBR repair pathways in cells of interest, the method comprising:
a) contacting the genome of cells of interest with an endonuclease to create blunt-ended double strand breaks (DSBs) at a predetermined location in the genome of the cells of interest, and
b) after the cells have been maintained in culture for sufficient time to allow repair of the DSBs by the cells' innate DNA repair machinery, determining whether the repaired DNA comprises an HR-specific DSBR signature sequence, an NHEJ-specific DSBR signature sequence, or an MMEJ DSBR signature sequence, in the region of the cells' genome spanning the DSB location,
wherein, if the repaired DNA comprises an HR-specific DSBR signature sequence then the HR DSBR pathway is active in the cells of interest, and if the repaired DNA comprises a NHEJ-specific DSBR signature sequence then the NHEJ DSBR pathway is active in the cells of interest, and if the repaired DNA comprises an MMEJ-specific DSBR signature sequence then the MMEJ DSBR pathway is active in the cells of interest.

27. The method of claim 26, wherein the endonuclease is a Cas9 endonuclease.

28. The method of claim 26, wherein step a) comprises transfecting the cells of interest with a vector comprising a Cas9 endonuclease expression cassette and a guide RNA (gRNA) cassette, wherein the gRNA is specific for the predetermined location in the genome.

29. The method of claim 26, wherein the predetermined location in the genome is in the within the AAVS1 safe-harbor site on human chromosome 19 (locus PPP1R12C).

30. The method of claim 29, wherein the predetermined location in the genome is between nucleotides 55115755 and 55115754 of human chromosome 19, GRCh38.p12.

31. The method of claim 26, wherein the predetermined location in the genome is within the AAVS1 safe-harbor site on chromosome 19 (locus PPP1R12C) of the human genome and wherein step a) comprises transfecting the cells of interest and the control cells with a vector comprising a Cas9 endonuclease expression cassette and a guide RNA (gRNA) cassette, wherein the gRNA is specific for the predetermined location AAVS1 safe harbor site.

32. The method of claim 28 or claim 31, wherein the vector is the pX330 vector.

33. The method of claim 26, wherein step a) further comprises transfecting the cells of interest with a donor template for homologous recombination, wherein the donor template comprises one or more mutations that are not present at the predetermined location in the genome of the cells of interest.

34. The method of claim 26, wherein step b) comprises determining the DNA sequences of the genomic DNA from the cells of interest using a next generation sequencing (NGS) method.

35. The method of claim 26, wherein step b) comprises determining DNA sequences resulting from more than 1,000 different repair events using a next generation sequencing (NGS) method.

36. The method of claim 26, wherein step b) comprises determining DNA sequences resulting from more than 10,000 different repair events using a next generation sequencing (NGS) method.

37. The method of claim 26, wherein step b) comprises determining DNA sequences resulting from more than 100,000 different repair events using a next generation sequencing (NGS) method.

38. The method of claim 26, wherein step b) comprises determining DNA sequences resulting from more than 200,000 different repair events using a next generation sequencing (NGS) method.

39. The method of claim 26, wherein in step b) the cells have been maintained in culture for about 3 days.

40. The method of claim 26, further comprising culturing the cells of interest for sufficient time to allow repair of the DSB by the cells' innate DNA repair machinery prior to performing step b).

41. The method of claim 40, wherein the cells are maintained in culture for about 3 days

42. The method of claim 26, wherein step b) comprises performing a PCR reaction to generate PCR products that that span the location of the DSBs in the genomic DNA from the cells of interest.

43. The method of claim 26, wherein step b) comprises performing a droplet digital PCR reaction to generate PCR products that that span the location of the DSBs in the genomic DNA from the cells of interest.

44. The method of claim 30, wherein step b) comprises performing a PCR reaction to generate PCR products that span the location of the DSBs in the genomic DNA from the cells of interest, and further comprises contacting the PCR products with (i) a MMEJ-specific probe comprising SEQ ID NO. 5, and/or (ii) a NHEJ-specific probe comprising SEQ ID NO. 6, and/or (ii) a HR-specific probe comprising SEQ ID NO. 7,wherein if the MMEJ-specific probe binds to the PCR product then the MMEJ DSBR pathway is active in the cells of interest, and if the NHEJ-specific probe binds to the PCR product then the NHEJ DSBR pathway is active in the cells of interest, and if the HR-specific probe binds to the PCR product then the HR DSBR pathway is active in the cells of interest.

45. The method of claim 31, wherein step b) comprises performing a PCR reaction to generate PCR products that span the location of the DSBs in the genomic DNA from the cells of interest, and further comprises contacting the PCR products with (i) a MMEJ-specific probe comprising SEQ ID NO. 5, and/or (ii) a NHEJ-specific probe comprising SEQ ID NO. 6, and/or (ii) a HR-specific probe comprising SEQ ID NO. 7, wherein if the MMEJ-specific probe binds to the PCR product then the MMEJ DSBR pathway is active in the cells of interest, and if the NHEJ-specific probe binds to the PCR product then the NHEJ DSBR pathway is active in the cells of interest, and if the HR-specific probe binds to the PCR product then the HR DSBR pathway is active in the cells of interest.

46. The method of any of claims 26-45, wherein the cells of interest are cancer cells.

47. The method of any of claims 26-45, wherein the cells of interest are peripheral blood mononuclear cells (PBMCs).

48. The method of any of claims 26-45, wherein the cells of interest are obtained from a biopsy sample.

49. The method of any of claims 26-45, wherein the cells of interest are obtained from a patient-derived xenograft (PDX).

50. The method of any of claims 26-45, wherein the cells of interest are human cells.

51. The method of any of claims 26-50, further comprising quantifying the relative usage of HR, NHEJ and MMEJ repair mechanisms in the cells of interest by quantifying the number of repair events or sequence reads comprising HR DSBR signature sequences, NHEJ DSBR signature sequences, and MMEJ DSBR signature sequences.

52. The method of any of claims 26-51, wherein the method is performed in the presence or absence of an inhibitor or candidate inhibitor of double strand break repair during the time that the DSB is generated and/or during the recovery time after DSB generation when DSBR would normally occur, to monitor the effect of that inhibitor or candidate inhibitor on the DSBR process and/or on the relative usage of the HR, NHEJ, and/or MMEJ pathways of DSBR.

53. A method of assessing the activity and/or usage of homologous recombination (HR), nonhomologous end-joining (NHEJ) and/or microhomology-mediated end-joining (MMEJ) DSBR repair pathways in cells of interest by sequencing, the method comprising:
(a) determining the DNA sequence of a genomic region spanning a repaired double strand break in a cell of interest (test DNA sequence), and
(b) comparing the test DNA sequence to a control DNA sequence, wherein the control DNA sequence comprises a wild-type version of the same genomic region that has not been subjected to a double strand break or to DSB repair,
wherein: (i) if the test DNA sequence comprises a 1 bp deletion relative to the control DNA sequence it has been repaired by nonhomologous end-joining (NHEJ), and (ii) if the test DNA sequence comprises a 12bp deletion with 5bp of microhomology (MH) relative to the control DNA sequence it has been repaired by MMEJ.

54. A method of assessing the activity and/or usage of homologous recombination (HR), nonhomologous end-joining (NHEJ) and/or microhomology-mediated end-joining (MMEJ) DSBR repair pathways in cells of interest by sequencing, the method comprising:
(a) determining the DNA sequence of a genomic region spanning a repaired double strand break (DSB) in a cell of interest (test DNA sequence), wherein the DSB was between nucleotides 55115755 and 55115754 of human chromosome 19, GRCh38.p12 within the AAVS1 safe-harbor site, and
(b) comparing the test DNA sequence to a control DNA sequence, wherein the control DNA sequence comprises a wild-type version of the same genomic region that has not been subjected to a double strand break or to DSB repair,
wherein: (i) if the test DNA sequence comprises a 1 bp deletion relative to the control DNA sequence it has been repaired by nonhomologous end-joining (NHEJ), and (ii) if the test DNA sequence comprises a 12 bp deletion with 5 bp of microhomology (MH) relative to the control DNA sequence it has been repaired by MMEJ.

55. A method of assessing the activity and/or usage of homologous recombination (HR), nonhomologous end-joining (NHEJ) and/or microhomology-mediated end-joining (MMEJ) DSBR repair pathways in cells of interest by PCR, the method comprising:
(a) amplifying a genomic region spanning a repaired double strand break (DSB) between nucleotides 55115755 and 55115754 of human chromosome 19, GRCh38.p12 (within the AAVS1 safe-harbor site) from a test cell of interest by PCR to generate test PCR products, and
(b) optionally amplifying the same genomic region that has not been subjected to a double strand break or to DSB repair from a control cell to generate control PCR products, and
(c) contacting the test PCR products and optionally the control PCR products with (i) a MMEJ-specific probe comprising SEQ ID NO. 5 and/or (ii) a NHEJ-specific probe comprising SEQ ID NO. 6, and/or (iii) a HR-specific probe comprising SEQ ID NO. 7.
wherein, if the MMEJ-specific probe binds to the test product PCR product then the MMEJ DSBR pathway is active in the cells of interest, and/or if the MMEJ-specific probe binds to the test product PCR product then the MMEJ DSBR pathway is active in the cells of interest, and/or if the HR-specific probe binds to the test product PCR product then the HR DSBR pathway is active in the cells of interest.

56. The method of claim 55, wherein the PCR is droplet digital PCR (ddPCR).

57. The method of claim 55, wherein in step (a) the genomic region spanning the repaired double strand break (DSB) is amplified using a forward primer comprising SEQ ID NO. 1 and a reverse primer comprising SEQ ID NO. 2.

58. The method of claim 56, wherein in step (a) the genomic region spanning the repaired double strand break (DSB) is amplified using a forward primer comprising SEQ ID NO. 1 and a reverse primer comprising SEQ ID NO. 2.

59. The method of any of claims 53-58, further comprising quantifying the relative usage of HR, NHEJ and MMEJ repair mechanisms in the cells of interest by quantifying the number of repair events or sequence reads comprising HR DSBR signature sequences, NHEJ DSBR signature sequences, and MMEJ DSBR signature sequences.

60. The method of any of claims 53-58, wherein the cells of interest were cultured in the presence or absence of an inhibitor or candidate inhibitor of double strand break repair during the time that the DSB is generated and/or during the recovery time after DSB generation when DSBR would normally occur, to monitor the effect of that inhibitor or candidate inhibitor on the DSBR process and/or on the relative usage of the HR, NHEJ, and/or MMEJ pathways of DSBR.

61. A kit for assessing the activity and/or usage of homologous recombination (HR), nonhomologous end-joining (NHEJ) and/or microhomology-mediated end-joining (MMEJ) DSBR repair pathways for repair of a double strand break (DSB) between nucleotides 55115755 and 55115754 of human chromosome 19, GRCh38.p12 (within the AAVS1 safe-harbor site), the kit comprising:
(a) a forward PCR primer comprising SEQ ID NO. 1,
(b) a reverse PCR primer comprising SEQ ID NO. 2,
(c) a MMEJ-specific probe comprising SEQ ID NO. 5,
(d) a NHEJ-specific probe comprising SEQ ID NO. 6, and optionally
(e) a HR-specific probe comprising SEQ ID NO. 7.

62. A kit for assessing the activity and/or usage of homologous recombination (HR), nonhomologous end-joining (NHEJ) and/or microhomology-mediated end-joining (MMEJ) DSBR repair pathways for repair of a double strand break (DSB) between nucleotides 55115755 and 55115754 of human chromosome 19, GRCh38.p12 (within the AAVS1 safe-harbor site), the kit comprising:
(a) a MMEJ-specific probe comprising SEQ ID NO. 5,
(b) a NHEJ-specific probe comprising SEQ ID NO. 6, and optionally
(c) a HR-specific probe comprising SEQ ID NO. 7.

63. A composition for assessing the activity and/or usage of homologous recombination (HR), nonhomologous end-joining (NHEJ) and/or microhomology-mediated end-joining (MMEJ) DSBR repair pathways for repair of a double strand break (DSB) between nucleotides 55115755 and 55115754 of human chromosome 19, GRCh38.p12 (within the AAVS1 safe-harbor site), the kit comprising:
(a) a forward PCR primer comprising SEQ ID NO. 1,
(b) a reverse PCR primer comprising SEQ ID NO. 2,
(c) a MMEJ-specific probe comprising SEQ ID NO. 5,
(d) a NHEJ-specific probe comprising SEQ ID NO. 6, and optionally
(e) a HR-specific probe comprising SEQ ID NO. 7.

64. A composition for assessing the activity and/or usage of homologous recombination (HR), nonhomologous end-joining (NHEJ) and/or microhomology-mediated end-joining (MMEJ) DSBR repair pathways for repair of a double strand break (DSB) between nucleotides 55115755 and 55115754 of human chromosome 19, GRCh38.p12 (within the AAVS1 safe-harbor site), the kit comprising:
(a) a MMEJ-specific probe comprising SEQ ID NO. 5,
(b) a NHEJ-specific probe comprising SEQ ID NO. 6, and optionally
(c) a HR-specific probe comprising SEQ ID NO. 7.

65. A composition or kit according to any of claims 61 to 64, further comprising:
(a) a WT probe comprising SEQ ID NO. 3, and/or
(b) a reference probe comprising SEQ ID NO. 6.

66. A composition or kit according to any of claims 61 to 65, wherein the primers and/or probes comprise a detectable moiety.

67. A composition or kit according to any of claims 61 to 65, wherein the primers and/or probes comprise a fluorescent label.

68. A composition or kit according to any of claims 61 to 65, wherein the primers and/or probes comprise a fluorophore having a fluorescence property that changes upon hybridization.

69. A composition or kit according to any of claims 61 to 65, wherein the primers and/or probes comprise a fluorophore and a quencher.
